(19) United States
(12) Patent Application Publication
(10) Pub. No.: US 2008/0196175 A1
(43) Pub. Date: Aug. 21, 2008

(54) Particular Dissymmetrical Cationic Polyazo Compounds, Compositions Comprising Them as Direct Dye, Process For Dyeing Keratin Fibres and Device Therefor

(76) Inventors: Andrew Greaves, Montevrain (FR); Herve David, La Verenne Saint Hilaire (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/791,457

(22) PCT Filed: Nov. 24, 2005
(86) PCT No.: PCT/EP05/13981

§ 371 Date: Nov. 26, 2007

Related U.S. Application Data
(60) Provisional application No. 60/634,527, filed on Dec. 10, 2004.

(30) Foreign Application Priority Data
Nov. 26, 2004 (FR) 0452785

(51) Int. Cl. A61K 8/49; C09B 44/10; C09B 44/16; A61Q 5/10; C09B 44/12; C09B 44/14

(52) U.S. Cl. 8/407; 534/608; 8/405

(57) ABSTRACT

The present invention concerns a new dissymmetrical cationic polyazo compounds of formula (I) Dye1-L-Dye2. The present invention also relates to dye compositions comprising such compounds as direct dye in a medium that is suitable for dyeing keratin fibres, and also to a process for dyeing keratin fibres using this composition and a multi-compartment device. The present invention provides direct dyes with which strong and varied shades that are resistant to external attacking factors such as bad weather and shampooing, for which there is no observation of any problem of change in colour over time.

The present invention relates to particular dissymmetrical cationic disazo compounds, to dye compositions comprising such compounds as direct dye in a medium that is suitable for dyeing keratin fibres, and also to a process for dyeing keratin fibres using this composition and a multi-compartment device.

It is known practice to dye keratin fibres, and in particular human keratin fibres such as the hair, with dye compositions containing direct dyes. These compounds are coloured and colouring molecules with affinity for the fibres. It is known practice, for example, to use direct dyes of the nitrobenzene type, anthraquinone dyes, nitropyridines and dyes of the azo, xanthene, acridine, azine or triarylmethane type.

These dyes are usually applied to the fibres, optionally in the presence of an oxidizing agent if it is desired to obtain simultaneous lightening of the fibres. Once the leave-in time has elapsed, the fibres are rinsed, optionally washed and dried.

The colorations resulting from the use of direct dyes are temporary or semi-permanent colorations since the nature of the interactions that bind the direct dyes to the keratin fibre, and their desorption from the surface and/or the core of the fibre, are responsible for their poor dyeing power and their poor relative resistance to washing or to perspiration.

Certain direct dyes may moreover have insufficient photostability properties.

An additional difficulty is also encountered which is associated with the fact that, in order to obtain a particular colour, it is necessary in most if not all cases to mix a plurality of dyes. However, not every dye has the same affinity for the fibre, which is reflected either in heterogeneous colorations or in changes in colour over time, for example after the fibres have been washed one or more times, exposed to sunlight, etc.

The aim of the present invention is to provide direct dyes that do not have the drawbacks of the existing direct dyes.

In particular, one of the aims of the present invention is to provide direct dyes with which strong and varied shades can be obtained that are resistant to external attacking factors (bad weather and shampooing), for which there is no observation of any problem of change in colour over time.

These aims and others are achieved by means of the present invention, one subject of which is thus dissymmetrical cationic polyazo compounds of formula (I) below:

Dye1-L-Dye2

in which formula:
Dye1 represents:

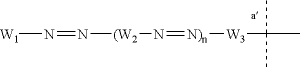

Dye2 represents:

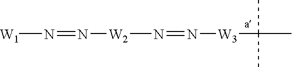

in which:
W₁, which are identical or different but are preferably identical, represent a heteroaromatic radical chosen from the following formulae:

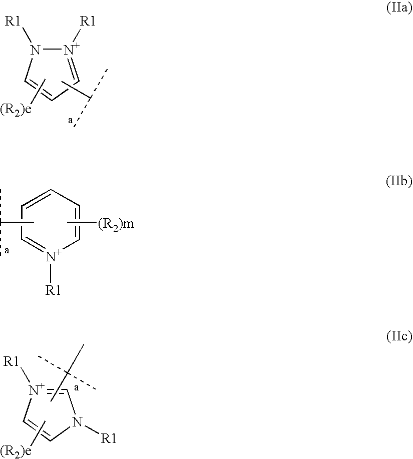

W₂, which may be identical or different, represent a carbon-based aromatic group having the following formula:

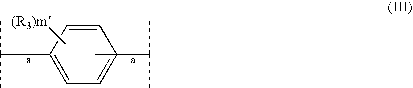

W₃, which may be identical or different, represent a carbon-based aromatic group having the following formula:

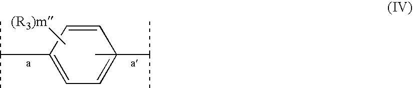

L represents a C₁-C₆₀, preferably C₂-C₄₀ and even more particularly C₂-C₂₀ saturated or unsaturated, linear or branched, cyclic or non-cyclic, (hetero)aromatic or non-(hetero)aromatic, optionally substituted hydrocarbon-based chain, optionally bearing at least one cationic charge, optionally interrupted with at least one hetero atom or group comprising at least one hetero atom, preferably oxygen, nitrogen or sulfur, L not comprising any diazo, nitro, nitroso or peroxide groups; L may be terminated at one and/or the other of its ends with at least one hetero atom or group comprising at least one hetero atom, preferably oxygen, nitrogen or sulfur;
n is 0 or 1;
R₁, independently of each other, represent:
   a linear or branched, saturated or unsaturated, aromatic or non-aromatic, optionally substituted C₁-C₁₆ hydrocarbon-based chain, which can form one or more 3- to 7-membered carbon-based rings, optionally interrupted with one or more hetero atoms chosen from oxygen, nitrogen and sulfur; R₁ not comprising any nitro, nitroso, peroxide or diazo bonds. R₁ is directly attached to the nitrogen atom (quaternized or non-quaternized) of the heteroaromatic ring via a carbon atom;
R₂, R₃ and R₄, independently of each other, represent:
   a linear or branched, saturated or unsaturated, aromatic or non-aromatic, optionally substituted C₁-C₁₆ hydrocarbon-based chain, which can form one or more 3- to 6-membered carbon-based rings, optionally interrupted with one or more hetero atoms or with one or more groups bearing at least one hetero atom, preferably chosen from oxygen and nitrogen;
   a hydroxyl group,
   a C₁-C₄ alkoxy group,
   a C₂-C₄ (poly)hydroxyalkoxy group; an alkoxycarbonyl group (RO—CO—) in which R represents a C₁-C₄ alkyl radical, an alkylcarbonyloxy radical (RCO—O—) in which R represents a C₁-C₄ alkyl radical;
   an amino group, an amino group substituted with one or more C₁-C₄ alkyl radicals, which may be identical or different, optionally bearing at least one hydroxyl group, the two alkyl radicals possibly forming, with the nitrogen atom to which they are attached, a 5- or 6-membered heterocycle optionally bearing another hetero atom identical to or different from nitrogen, for example oxygen or sulfur; an alkylcarbonylamino group (RCO—NR—) in which the radicals R, independently of each other, represent a C₁-C₄ alkyl radical; a carbamoyl group ((R)₂N—CO) in which the radicals R, independently of each other, which may be identical or different, represent a hydrogen atom or a C₁-C₄ alkyl radical; a ureido group ((R)₂N—CO—NR′—) in which the radicals R and R′, independently of each other, represent a hydrogen atom or a C₁-C₄ alkyl radical; a sulfonamide group ((R)₂N—SO₂—) in which the radicals R, independently of each other, represent a hydrogen atom or a C₁-C₄ alkyl radical; an alkylsulfonylamino group (RSO₂—NR′—) in which the radicals R and R′, independently of each other, represent a hydrogen atom or a C₁-C₄ alkyl radical; a guanidinium group ((R′)₂N—C(═NH₂⁺)—NR—) in which the radicals R and R′, independently of each other, represent a hydrogen atom or a C₁-C₄ alkyl radical;
   a nitro group; a cyano group; a halogen atom, preferably chlorine or fluorine;
   two radicals R₂ or two radicals R₃ or two radicals R₄, borne by adjacent carbon atoms, can form, with the carbon atom to which each is attached, a fused ring:
m represents an integer between 0 and 4;
m′ represents an integer between 0 and 4;
m″ represents an integer between 0 and 4;
e is an integer between 0 and 2;
the bond a derived from formula IIa, IIb, IIc, III or IV links the groups W₁, W₂ and W₃ to the azo group;
in the case of formulae IIa and IIc, and when two radicals R₂ borne by two adjacent carbon atoms form an aromatic ring, the bond a may link the group W₁ to the azo group via the said aromatic ring;
the bond a′ derived from formula IV links the group W₃ to the linker L;
the electrical neutrality of the compounds being ensured by means of one or more cosmetically acceptable, identical or different anions An.

A subject of the present invention is also dye compositions comprising such compounds, as direct dyes, in a medium that is suitable for dyeing keratin fibres.

The invention also relates to a process for dyeing keratin fibres which consists in placing a composition according to the invention in contact with the said wet or dry fibres, for a time that is sufficient to obtain the desired effect.

Finally, the invention relates to a multi-compartment device comprising, in a first compartment, the composition according to the invention, and, in a second compartment, an oxidizing composition.

It has been found that the compounds of formula (I) as defined above show good fastness with respect to external agents such as, especially, shampoos.

Furthermore, these compounds show good photostability.

Moreover, the compounds of formula (I) allow the range of colours obtained to be enhanced, since the members of formula (I) either side of L may be different in colour.

However, other characteristics and advantages of the present invention will emerge more clearly on reading the description and the examples that will be presented.

In the text hereinbelow, unless otherwise indicated, the limits delimiting a range of values are included in that range of values.

Moreover, the keratin fibres forming the subject of the treatment according to the invention are preferably human keratin fibres, in particular the hair.

For the purposes of the present invention, and unless otherwise indicated:
The compounds of formula (I) are said to be dissymmetrical when the groups Dye1 and Dye2 are different; in other words this signifies that there is no axis or plane of symmetry passing through L, the said axis or plane of symmetry being coincident with or perpendicular to the main chain of L.
The members of formula (I) either side of the group L, also, are said to be different when the groups W₁ relative to one another, and/or the groups W₂ relative to one another, and/or the groups W₃ relative to one another, are alternatively (a) different in structure, (b) the same in structure but with different substituents, (c) or the same in structure and with the same substituents, the latter not being in the same positions from one group to another.
an alkyl radical may be monovalent or divalent, and is linear or branched,
an alkyl radical or the alkyl part of a radical is said to be substituted when it comprises at least one substituent chosen from the following groups:
   n hydroxyl,
   C₁-C₄ alkoxy, C₂-C₄ (poly)hydroxyalkoxy,
   amino, amino substituted with one or more C₁-C₄ alkyl groups, which may be identical or different, optionally bearing at least one hydroxyl group, the said alkyl radicals possibly forming, with the nitrogen to which they are attached, a 5- or 6-membered hetero-cycle optionally comprising at least one other nitrogen or non-nitrogen hetero atom,
an aryl or heteroaryl radical or the aryl or heteroaryl part of a radical is said to be substituted when it comprises at least one substituent borne by a carbon atom, chosen from
   C₁-C₁₆ and preferably C₁-C₈ alkyl radical, optionally substituted with one or more radicals chosen from hydroxyl, C₁-C₂ alkoxy, C₂-C₄ (poly)hydroxyalkoxy, acylamino, amino substituted with two identical or different C₁-C₄ alkyl radicals, optionally bearing at least one hydroxyl group, or the two radicals possibly forming, with the nitrogen atom to which they are attached, a 5- to 7-membered and preferably 5- or 6-membered heterocycle, optionally comprising another nitrogen or non-nitrogen hetero atom;
   a halogen atom such as chlorine, fluorine or bromine;
   a hydroxyl group;
   a C₁-C₂ alkoxy radical; a C₂-C₄ (poly)hydroxy-alkoxy radical;
   an amino radical; an amino radical substituted with one or two identical or different C₁-C₄ alkyl radicals optionally bearing at least one hydroxyl group, or amino with two optionally substituted C₁-C₂ alkyl radicals;
   an acylamino radical (—NR—COR′) in which the radical R is a hydrogen atom, a C₁-C₄ alkyl radical optionally bearing at least one hydroxyl group and the radical R′ is a C₁-C₂ alkyl radical; a carbamoyl radical ((R)₂N—CO—) in which the radicals R, which may be identical or different, represent a hydrogen atom or a C₁-C₄ alkyl radical optionally bearing at least one hydroxyl group; an alkylsulfonylamino radical (R′SO₂—NR—) in which the radical R represents a hydrogen atom or a C₁-C₄ alkyl radical optionally bearing at least one hydroxyl group and the radical R′ represents a C₁-C₄ alkyl radical or a phenyl radical; an aminosulfonyl radical ((R)₂N—SO₂—) in which the radicals R, which may be identical or different, represent a hydrogen atom or a C₁-C₄ alkyl radical optionally bearing at least one hydroxyl group,
the cyclic or heterocyclic part of a non-aromatic radical is said to be substituted when it comprises at least one substituent borne by a carbon atom, chosen from the following groups:
   hydroxyl,
   C₁-C₄ alkoxy, C₂-C₄ (poly) hydroxyalkoxy,
   alkylcarbonylamino((RCO—NR′—) in which the radical R′ is a hydrogen atom or a C₁-C₄ alkyl radical optionally bearing at least one hydroxyl group and the radical R is a C₁-C₂ alkyl radical, an amino radical substituted with two identical or different C₁-C₄ alkyl groups optionally bearing at least one hydroxyl group, the said alkyl radicals possibly forming, with the nitrogen atom to which they are attached, a 5- or 6-membered heterocycle optionally comprising at least one other nitrogen or non-nitrogen hetero atom;
when a ring does not bear the maximum number of substituents, then the unsubstituted position(s) bear(s) a hydrogen atom.

As indicated previously, a first subject of the invention consists of compounds corresponding to the abovementioned formula (I).

As regards the radicals R₁, they represent, independently of each other, a C₁-C₁₅ alkyl radical; a C₂-C₆ monohydroxyalkyl radical; a C₂-C₆ polyhydroxyalkyl radical; a (C₁-C₆)alkoxy(C₂-C₆)alkyl radical; an optionally substituted aryl radical, such as phenyl; an optionally substituted arylalkyl radical, such as benzyl; a C₂-C₆ amidoalkyl radical; a C₂-C₆ aminoalkyl radical in which the amine is substituted with two optionally substituted C₁-C₄ alkyl radicals, which may be identical or different. Furthermore, the radical R₁ is such that the atom directly linked to the nitrogen atom is a carbon atom.

Preferably, R₁ represents a C₁-C₆ alkyl radical, a C₂-C₆ monohydroxyalkyl radical; a C₂-C₆ polyhydroxyalkyl radical; a (C₁-C₆)alkoxy(C₂-C₆)alkyl radical; a phenyl radical optionally substituted with at least one chlorine atom, a hydroxyl group, a group RCO—NH— in which R represents a C₁-C₄ alkyl radical, an amino radical substituted with two C₁-C₄ alkyl radicals, which may be identical or different; a benzyl radical; a C₁-C₆ aminoalkyl radical; a C₁-C₆ aminoalkyl radical in which the amine is substituted with two C₁-C₄ alkyl radicals, which may be identical or different.

The radicals R₂, R₃ and R₄, independently of each other, more particularly represent:
a hydrogen atom;
an optionally substituted C₁-C₁₆ and preferably C₁-C₈ alkyl radical;
a halogen atom such as chlorine, fluorine or bromine;
a hydroxyl group;
a C₁-C₂ alkoxy radical; a C₂-C₄ (poly)hydroxyalkoxy radical;
an amino radical, an amino radical substituted with one or two C₁-C₄ alkyl radicals, which may be identical or different, optionally bearing at least one hydroxyl or C₁-C₄ dialkylamino group, the two alkyl radicals possibly forming, with the nitrogen atom to which they are attached, a saturated or unsaturated, optionally substituted 5- or 6-membered heterocycle optionally bearing another hetero atom identical to or different from nitrogen;
an alkylcarbonylamino radical (RCO—NR′—) in which the radical R represents a C₁-C₄ alkyl radical and the radical R′ represents a hydrogen atom or a C₁-C₄ alkyl radical; a carbamoyl radical ((R)₂N—CO—) in which the radicals R, which may be identical or different, represent a hydrogen atom or a C₁-C₄ alkyl radical optionally bearing at least one hydroxyl group; an alkylsulfonylamino radical (R′SO₂—NR—) in which the radical R represents a hydrogen atom or a C₁-C₄ alkyl radical optionally bearing at least one hydroxyl group, and the radical R′ represents a C₁-C₄ alkyl radical; an aminosulfonyl radical ((R)₂N—SO₂—) in which the radicals R, which may be identical or different, represent a hydrogen atom or a C₁-C₄ alkyl radical optionally bearing at least one hydroxyl group; a ureido radical (N(R)₂—CO—NR′—) in which the radicals R and R′, which may be identical or different, represent a hydrogen atom or a C₁-C₄ alkyl radical.

More preferentially, the radicals R₂, R₃ and R₄, independently of each other, represent:
a hydrogen atom;
a C₁-C₄ alkyl radical optionally substituted with one or more radicals chosen from hydroxyl, acylamino and amino radicals substituted with two identical or different C₁-C₂ alkyl radicals, optionally bearing at least one hydroxyl group, or a C₁-C₂ alkoxy radical;
an amino radical; an amino radical substituted with one or two C₁-C₃ alkyl radicals, which may be identical or different, optionally bearing at least one hydroxyl group, the two alkyl radicals possibly forming, with the nitrogen atom to which they are attached, a saturated or unsaturated, optionally substituted 5- or 6-membered heterocycle optionally bearing another hetero atom identical to or different from nitrogen; an acylamino radical; a carbamoyl radical; a sulfonylamino radical;
a hydroxyl radical; a C₁-C₂ alkoxy radical;
a chlorine atom.

According to one preferred variant of the invention, the radicals R₂, R₃ and R₄, independently of each other, represent:
a hydrogen atom;
a methyl, ethyl, propyl, 2-hydroxyethyl, methoxy, ethoxy, 2-hydroxyethyloxy, 3-hydroxypropyloxy or 2-methoxyethyl radical;
a sulfonylamino radical; an amino, methylamino, dimethylamino, 2-hydroxyethylamino or 3-hydroxy-propylamino radical or an acylamino radical; a hydroxyl radical; a pyrrolidine, piperazine, piperidine or homopiperazine radical;
a chlorine atom.

According to a second preferred variant of the invention, two radicals R₂ borne by adjacent carbon atoms may form, together with the carbon atom to which each is attached, a fused ring optionally substituted preferably with a hydrogen; a methyl radical; a hydroxyl radical; a methoxy radical; an amino radical; a methylamino radical; a dimethylamino radical; a pyrrolidine radical; a sulfonylamino radical.

According to a third preferred variant of the invention, two radicals R₃ borne by adjacent carbon atoms can form, together with the carbon atom to which each is attached, a fused ring optionally substituted preferably with a hydrogen; a methyl radical; a hydroxyl radical; a methoxy radical; an amino radical; a methylamino radical; a dimethylamino radical; a pyrrolidine radical; a sulfonylamino radical.

According to one particular document of the invention, the linker L is a chain that isolates each of the chromophores so as to stop the electron delocalization of each of the chromophores.

According to a first variant of the invention, in formula (I), L may be represented by formula (V) below:

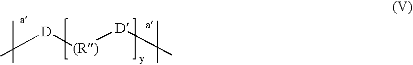

in which:
D and D′, independently of each other, represent a linear or branched C₁-C₁₄ hydrocarbon-based bond, which can form one or more 5- to 8-membered carbon-based rings, and which may be saturated or unsaturated, optionally interrupted with one or more hetero atoms or with one or more groups bearing at least one hetero atom, preferably chosen from oxygen and nitrogen; D and/or D′ are linked to the ammonium atom via a carbon atom;
the bond a′ links the arms D and D′ to the groups W₃, preferably via an oxygen atom or a nitrogen atom;
y is greater than or equal to 1 and preferably equal to 1 or 2;
the group R″ representing

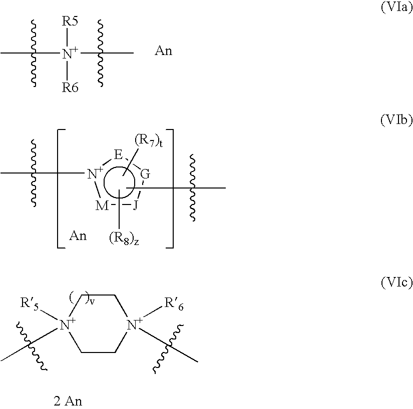

R₅, R₆, R′₅ and R′₆, independently of each other, represent a C₁-C₁₅ alkyl radical; a C₁-C₆ monohydroxyalkyl radical; a C₂-C₆ polyhydroxyalkyl radical; a (C₁-C₆)alkoxy(C₁-C₆)alkyl radical; an aryl radical such as phenyl; an arylalkyl radical such as benzyl; a C₁-C₆ amidoalkyl radical; a C₁-C₆ aminoalkyl radical; a C₁-C₆ aminoalkyl radical in which the amine is substituted with one or more identical or different C₁-C₄ alkyl, (C₁-C₆)alkylcarbonyl, acylamino or (C₁-C₆)alkylsulfonyl radicals;
R₅ and R₆ may form, together with the nitrogen atom to which they are attached, a 5-, 6- or 7-membered saturated cationic ring that may contain one or more hetero atoms, the cationic ring possibly being substituted with a halogen atom, a hydroxyl radical, a C₁-C₆ alkyl radical, a C₁-C₆ monohydroxyalkyl radical, a C₂-C₆ polyhydroxyalkyl radical, a C₁-C₆ alkoxy radical, an amido radical, a (C₁-C₆)alkyl-carbonyl radical, a thio radical, a C₁-C₆ thioalkyl radical, a (C₁-C₆)alkylthio radical, an amino radical or an amino radical substituted with one or more identical or different C₁-C₆ alkyl, (C₁-C₆)alkyl-carbonyl, acylamino or (C₁-C₆)alkylsulfonyl radicals; and preferably unsubstituted;
R₅ or R₆ may form, with D or D′, a 5-, 6- or 7-membered saturated cationic ring that may contain one or more hetero atoms, the cationic ring possibly being substituted with a halogen atom, a hydroxyl radical, a C₁-C₆ alkyl radical, a C₁-C₆ monohydroxy-alkyl radical, a C₂-C₆ polyhydroxyalkyl radical, a C₁-C₆ alkoxy radical, an amido radical, a (C₁-C₆)alkyl-carbonyl radical, a thio radical, a C₁-C₆ thioalkyl radical, a (C₁-C₆)alkylthio radical, an amino radical or an amino radical substituted with one or more identical or different C₁-C₆ alkyl, (C₁-C₆)alkyl-carbonyl, acylamino or (C₁-C₆)alkylsulfonyl radicals; and preferably unsubstituted;
the ring members E, G, J and M, which may be identical or different, represent a carbon, oxygen, sulfur or nitrogen atom to form a pyrazolium, imidazolium, triazolium, oxazolium, isoxazolium, thiazolium or isothiazolium ring,
R₇ represents a C₁-C₆ alkyl radical; a C₁-C₆ mono-hydroxyalkyl radical, a C₂-C₆ polyhydroxyalkyl radical, a (C₁-C₆)alkoxy(C₁-C₆)alkyl radical, a C₂-C₆ carbamylalkyl radical, a (C₁-C₆)alkylcarboxy(C₁-C₆)alkyl radical or a benzyl radical; it being understood that the radical R₇ is borne by a nitrogen atom,
R₈, which may be identical or different, represent a hydrogen or halogen atom, a C₁-C₆ alkyl radical, a C₁-C₆ monohydroxyalkyl radical, a C₂-C₆ polyhydroxyalkyl radical, a hydroxyl radical, a C₁-C₆ alkoxy radical, an amido radical, a carboxyl radical, a C₁-C₆ alkylcarbonyl radical, a C₁-C₆ thioalkyl radical, a (C₁-C₆)alkylthio radical, an amino radical disubstituted with a (C₁-C₆)alkyl, (C₁-C₆)alkylcarbonyl or (C₁-C₆)alkylsulfonyl radical, a benzyl radical, a phenyl radical optionally substituted with one or more radicals chosen from methyl, hydroxyl, amino and methoxy radicals, it being understood that the radicals R₈ are borne by a carbon atom,
An represents an organic or mineral anion or mixture of anions;
z is an integer between 1 and 3;
t is equal to 0 or 1; when t=0, then D′ is linked to the nitrogen atom via a carbon atom,
v is an integer equal to 1 or 2,
y is equal to 0 or 1.

Preferably, the linker represented by formula (V) is attached to the chromophores via a nitrogen or oxygen atom.

According to one particular embodiment of formula (VIa), R₅ and R₆, independently of each other, are preferably chosen from a C₁-C₆ alkyl radical, a C₁-C₄ monohydroxyalkyl radical, a C₂-C₄ polyhydroxyalkyl radical, a (C₁-C₆)alkoxy(C₂-C₄)alkyl radical, a C₂-C₆ amidoalkyl radical or a C₂-C₆ dimethylaminoalkyl radical.

Even more preferably, R₅ and R₆, independently of each other, represent a methyl, ethyl or 2-hydroxy-ethyl radical.

According to this variant, D and D′, separately, are preferably a C₁-C₆ alkyl chain that may be substituted, and that is preferably unsubstituted.

According to one particular embodiment of formula (VIb), the ring members E, G, J and M form an imidazolium, pyrazolium, oxazolium or thiazolium ring. In accordance with one even more preferred embodiment of the invention, formula (VIb) represents an imidazolium ring.

According to this particular embodiment, z and t are equal to 0.

In accordance with this embodiment, D and D′ represent a linear or branched, preferably linear, C₁-C₆ alkyl chain that may be substituted, and that is preferably unsubstituted.

According to one preferred embodiment of formula (VIc), R′₅ and R′₆ have the same meanings as R₅ and R₆, independently of these two radicals.

According to this variant, D and D′, separately, are preferably a C₁-C₆ alkyl chain that may be substituted, and that is preferably unsubstituted.

Preferably, the coefficient v is equal to 1.

According to a second variant of the invention, in formula (I), L represents a C₁-C₆₀, preferably C₂-C₄₀ and preferably C₂-C₂₀ linear or branched divalent hydrocarbon-based chain, to form one or more optionally substituted, optionally aromatic 3- to 7-membered carbon-based rings, this chain being optionally substituted, optionally interrupted with one or more hetero atoms or with one or more groups bearing a hetero atom, preferably chosen from oxygen, nitrogen and sulfur; L not comprising any peroxide, nitro, diazo or nitroso groups. Finally, L does not bear a cationic charge.

According to one more particular embodiment, L is a hydrocarbon-based chain that isolates each of the two members of formula (I) so as to stop electron delocalization.

Examples of linkers L that may be mentioned include alkylene radicals (CₙH₂ₙ) containing from 1 to 60 carbon atoms, preferably from 2 to 40 carbon atoms and more preferentially from 2 to 20 carbon atoms, optionally substituted and/or interrupted with one or more hetero atoms chosen from oxygen, nitrogen and sulfur and/or from an SO₂ or CO group. These alkylene radicals are, for example, propylene, butylene, pentylene, hexylene, etc.

Even more preferably, L is a linear or branched C₂-C₄₀ and preferably C₂-C₂₀ hydrocarbon-based chain, optionally substituted and/or interrupted with one or more hetero atoms chosen from oxygen, nitrogen and sulfur.

Examples of linkers L that may also be mentioned include the alkylene radicals as defined above, interrupted with a divalent (hetero)aryl radical.

According to this variant, L may be represented by:
C₁-C₂₀ alkyl-(hetero)aryl-C₁-C₂₀ alkyl and more advantageously:
C₁-C₁₀ alkyl-(hetero)aryl-C₁-C₁₀ alkyl.

The (hetero)arylene radicals are, for example, phenylene or naphthylene, phenanthrylene, triazinyl, pyrimidinyl, pyridyl, pyridazinyl or quinoxalinyl.

It should be noted that L advantageously represents an alkylene radical interrupted with a (hetero)arylene group, optionally substituted and/or interrupted with one or more hetero atoms chosen from oxygen and nitrogen, and/or from one or more SO₂ or CO groups.

As examples of alkylene radicals interrupted with a (hetero)arylene group, mention may be made of the following radicals:

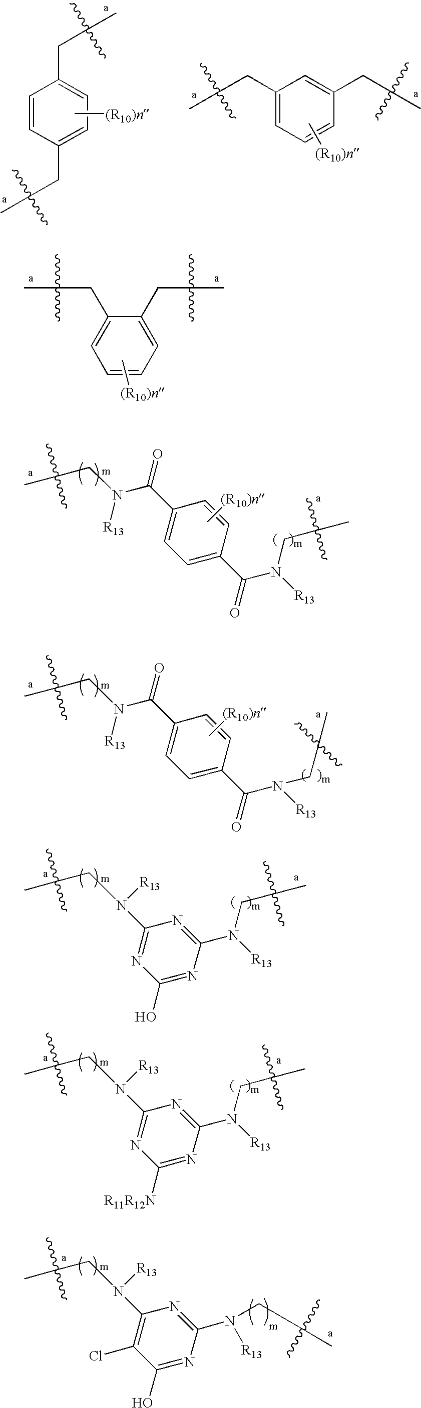

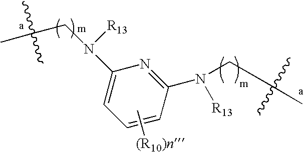

in which:
R₁₀ has the same definition as R₄,
R₁₁ and R₁₂ represent, independently of each other, a hydrogen atom or a linear or branched C₁-C₈ alkyl radical, optionally substituted with one or more hydroxyl, C₁-C₂ alkoxy, C₂-C₄ (poly)hydroxyalkoxy, amino, C₁-C₂ (di)alkylamino or optionally substituted aryl radicals,
R₁₃ radicals represent, independently of each other, a hydrogen atom or an optionally substituted C₁-C₄ alkyl radical,
m represents an integer between 1 and 6,
n″ represents an integer between 0 and 4,
n′″ represents an integer between 0 and 3.

Preferably, in the context of this variant, L represents

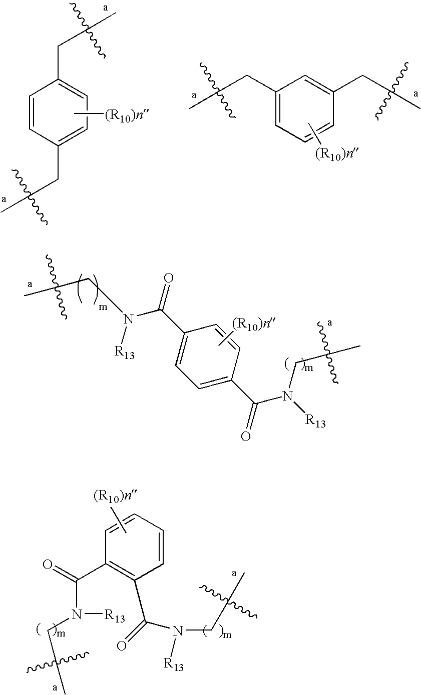

the radicals R₁₀, R₁₃, m and n″ being defined as above.

Linkers L that may also be mentioned include the triazines described in WO 03/029359, the alkylenes cited in U.S. Pat. No. 5,708,151 and the alkyl-aryl-alkyls cited in U.S. Pat. No. 5,708,151.

Preferably, L represents a linear or branched C₁-C₁₀ alkylene radical, optionally interrupted with one or more hetero atoms, chosen from oxygen, nitrogen and sulfur, optionally substituted with one or more radicals chosen from hydroxyl, C₁-C₂ alkoxy, C₁-C₂ dialkylamino, alkylcarbonyl (R—CO—) in which the radical R represents a C₁-C₄ alkyl radical, a carbamoyl group ((R)₂N—CO—) in which the radicals R, independently of each other, represent a hydrogen atom or an alkylsulfonyl radical (R—SO₂—) in which the radical R represents a C₁-C₄ alkyl radical.

More preferably, the linker L represents a C₁-C₁₀ alkylene radical, which is preferably linear, optionally interrupted with one or more hetero atoms chosen from oxygen, nitrogen and sulfur, and optionally substituted with one or more radicals chosen from hydroxyl and C₁-C₂ alkoxy radicals, and an alkylsulfonyl radical (R—SO₂—) in which the radical R represents a C₁-C₄ alkyl radical.

The linker L preferably represents a C₁-C₁₀ alkylene radical.

In formula (I), An is an organic or mineral anion or mixture of anions chosen, for example, from halides such as chlorides, bromides, fluorides and iodides; hydroxides; sulfates; hydrogen sulfates; (C₁-C₆)alkyl sulfates, for instance methyl sulfate or ethyl sulfate; phosphates; carbonates; hydrogen carbonates, perchlorates; acetates; tartrates; citrates, oxalates; (C₁-C₆)alkylsulfonates such as methylsulfonate; arylsulfonates that are unsubstituted or substituted with a C₁-C₄ alkyl radical, for instance a 4-tolylsulfonate.

According to one preferred embodiment of the invention, the compounds correspond more particularly to the following formulae:

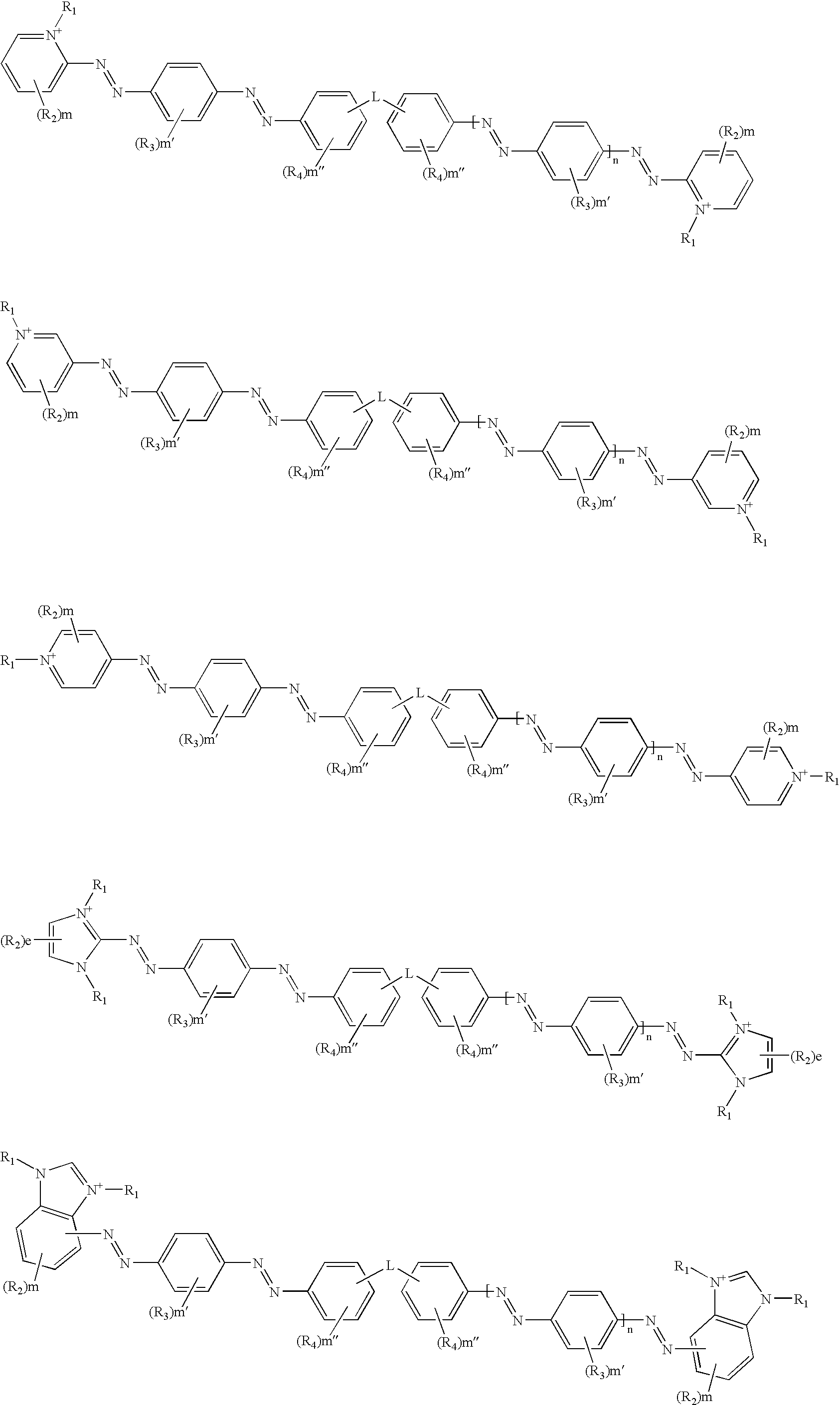

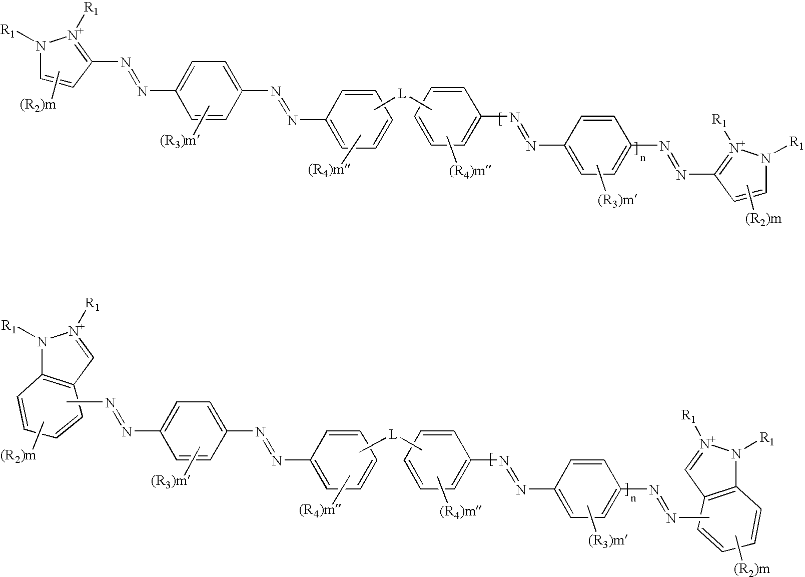

in which formulae:
R₁, R₂, R₃, R₄, L, n, m, m′ and m″ have been defined above; the same is true of their preferred values,
the electrical neutrality of the molecule being respected by means of the presence of one or more cosmetically acceptable anions An as defined above.

These compounds may be obtained especially from similar preparation processes described, for example, in documents BE 662 856, GB 932 022, U.S. Pat. No. 3,173,907 and U.S. Pat. No. 5,708,151.

According to a first embodiment, the process for synthesizing the compounds used in the invention may consist in performing the following steps:

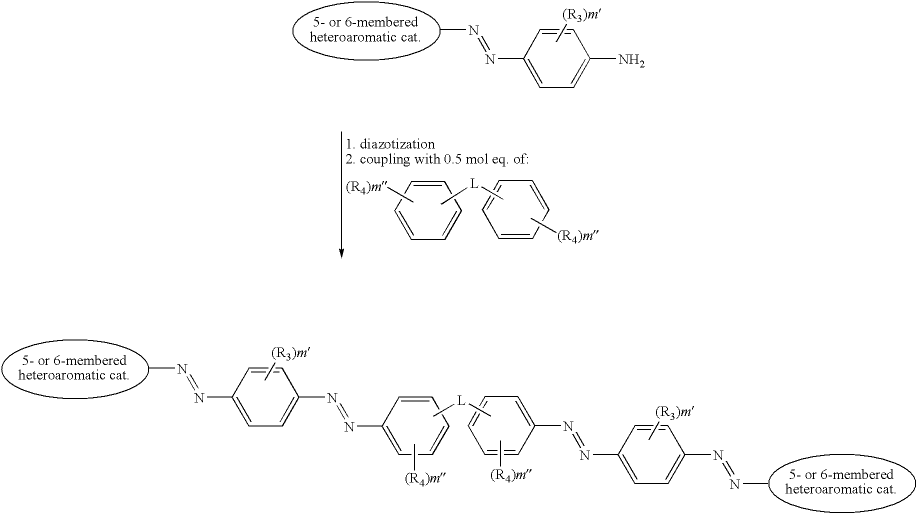

According to this process, a first step of diazotization of a 5- or 6-membered cationic heteroaromatic amine is performed in a manner known to those skilled in the art.

Thus, the said amine is placed in contact with sodium nitrite or nitrosylsulfuric acid. This reaction usually takes place at a temperature of between −15° C. and 30° C. and preferably between −10° C. and 20° C. and at a pH of between 0 and 12.

The reaction conventionally takes place in the presence of a suitable solvent, among which mention may be made of water, alcohols and especially aliphatic alcohols containing up to 4 carbon atoms, organic acids, for example a carboxylic or sulfonic acid containing up to 10 carbon atoms, and/or mineral acids such as hydrochloric acid or sulfuric acid.

Once the reaction has been performed, the product obtained is coupled with a compound of the type W₃-L-W₃, with W₃ advantageously representing an optionally substituted aromatic nucleus. The synthesis of these compounds is known to those skilled in the art (see for example Org. Lett. 2002, 4(4), 581-584; Org. Lett. 2003, 5(6), 793-796).

This reaction is usually performed in the presence of a solvent, which may be the solvent from the preceding step.

The temperature is conventionally between −15° C. and 30° C. and preferably between −10° C. and 20° C. at a pH preferably of between 0 and 8.

The product may be isolated via techniques known to those skilled in the art (precipitation, evaporation, etc.).

According to a second embodiment, the process for synthesizing the compounds used in the invention may consist in performing the following steps:

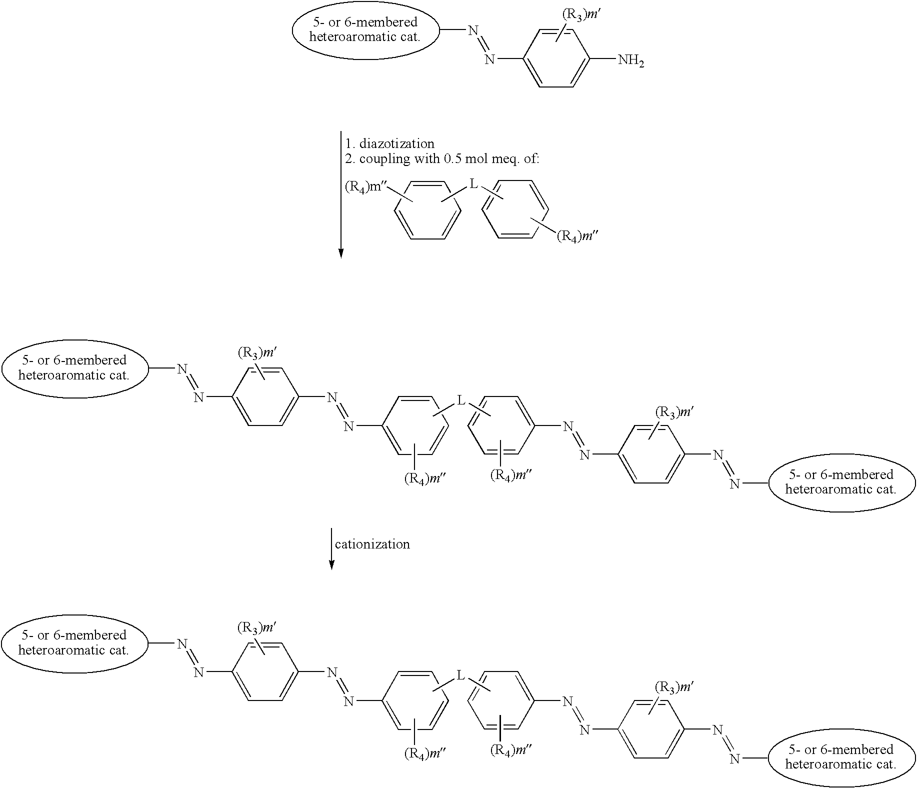

According to this process, a first step of diazotization of a cationizable heteroaromatic amine is performed in a manner known to those skilled in the art. The conditions for performing such a step have been summarized previously.

Once the reaction has been performed, the product obtained is coupled with a compound of the type W₃-L-W₃, with W₃ advantageously representing an optionally substituted aromatic nucleus. The conditions for performing such a step have been summarized previously.

The resulting product is then quaternized in a usual manner. For example, the product obtained may be placed in contact with an alkyl sulfate such as dimethyl sulfate, diethyl sulfate or dipropyl sulfate, or an alkyl halide or alkylaryl halide such as iodomethane, iodoethane, 2-bromoethanol or benzyl bromide, in the presence of a polar or apolar, protic or aprotic solvent such as dichloromethane, toluene, ethyl acetate or water, at the spontaneous or alkaline pH. The temperature is usually between 10° C. and 180° C. and preferably between 20° C. and 100° C.

The product may be isolated via the techniques known to those skilled in the art (precipitation, evaporation, etc.).

According to a third embodiment, the process for synthesizing the compounds used in the invention may consist in performing the following steps:

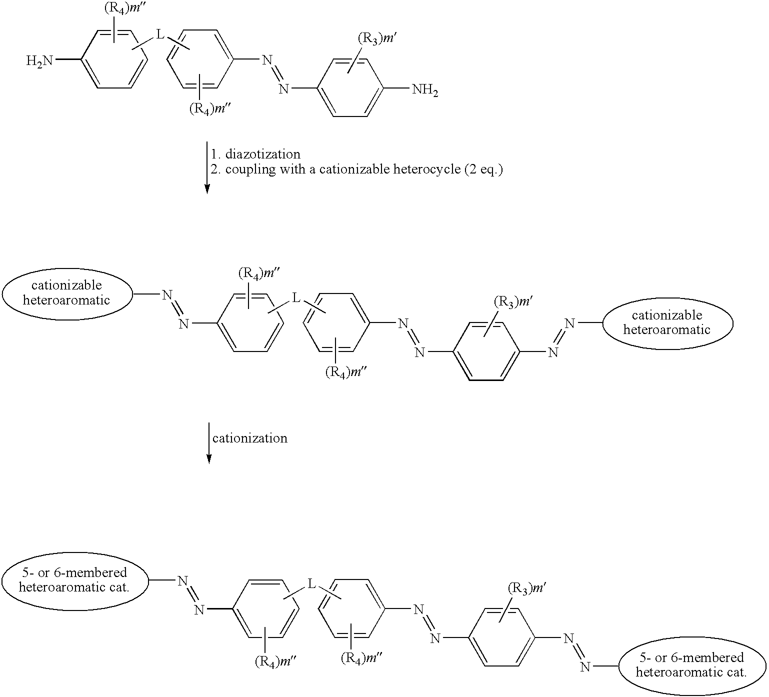

The conditions for performing such a synthesis have been summarized previously.

Another subject of the present invention consists of a composition comprising, in a medium that is suitable for dyeing keratin fibres, as direct dye, at least one dissymmetrical cationic polyazo compound of formula (I) below:

Dye1-L-Dye2

in which formula:
Dye1 represents:

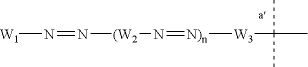

Dye2 represents:

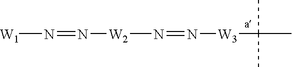

in which:
W₁, which may be identical or different but are preferably identical, represent a heteroaromatic radical chosen from the following formulae:

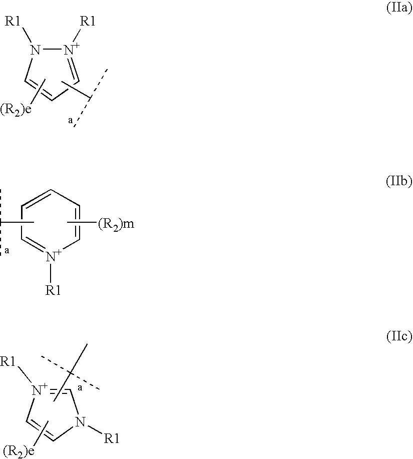

W₂, which may be identical or different, represent a carbon-based aromatic group having the following formula:

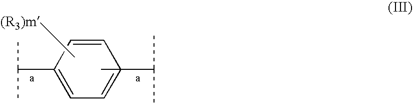

W₃, which may be identical or different, represent a carbon-based aromatic group having the following formula:

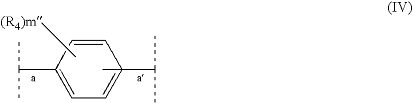

L represents a C₁-C₆₀, preferably C₂-C₄₀ and even more particularly C₂-C₂₀ saturated or unsaturated, linear or branched, cyclic or non-cyclic, (hetero)aromatic or non-(hetero)aromatic, optionally substituted hydrocarbon-based chain, optionally bearing at least one cationic charge, optionally interrupted with at least one hetero atom or group comprising at least one hetero atom, preferably oxygen, nitrogen or sulfur, L not comprising any diazo, nitro, nitroso or peroxide groups; L may be terminated at one and/or the other of its ends with at least one hetero atom or group comprising at least one hetero atom, preferably oxygen, nitrogen or sulfur;
n is 0 or 1;
R₁, independently of each other, represent:
   a linear or branched, saturated or unsaturated, aromatic or non-aromatic, optionally substituted C₁-C₁₆ hydrocarbon-based chain, which can form one or more 3- to 7-membered carbon-based rings, optionally interrupted with one or more hetero atoms chosen from oxygen, nitrogen and sulfur; R₁ not comprising any nitro, nitroso, peroxide or diazo bonds. R₁ is directly attached to the nitrogen atom (quaternized or non-quaternized) of the heteroaromatic ring via a carbon atom;
R₂, R₃ and R₄, independently of each other, represent:
   a linear or branched, saturated or unsaturated, aromatic or non-aromatic, optionally substituted C₁-C₁₆ hydrocarbon-based chain, which can form one or more 3- to 6-membered carbon-based rings, optionally interrupted with one or more hetero atoms or with one or more groups bearing at least one hetero atom, preferably chosen from oxygen and nitrogen;
   a hydroxyl group,
   a C₁-C₄ alkoxy group,
   a C₂-C₄ (poly)hydroxyalkoxy group; an alkoxycarbonyl group (RO—CO—) in which R represents a C₁-C₄ alkyl radical, an alkylcarbonyloxy radical (RCO—O—) in which R represents a C₁-C₄ alkyl radical;
   an amino group, an amino group substituted with one or more C₁-C₄ alkyl radicals, which may be identical or different, optionally bearing at least one hydroxyl group, the two alkyl radicals possibly forming, with the nitrogen atom to which they are attached, a 5- or 6-membered heterocycle optionally bearing another hetero atom identical to or different from nitrogen, for example oxygen or sulfur; an alkylcarbonylamino group (RCO—NR—) in which the radicals R, independently of each other, represent a C₁-C₄ alkyl radical; a carbamoyl group ((R)₂N—CO) in which the radicals R, independently of each other, which may be identical or different, represent a hydrogen atom or a C₁-C₄ alkyl radical; a ureido group ((R)₂N—CO—NR′—) in which the radicals R and R′ independently of each others represent a hydrogen atom or a C₁-C₄ alkyl radical; a sulfonamide group ((R)₂N—SO₂—) in which the radicals R, independently of each other, represent a hydrogen atom or a C₁-C₄ alkyl radical; an alkylsulfonylamino group (RSO₂—NR′—) in which the radicals R and R′, independently of each other, represent a hydrogen atom or a C₁-C₄ alkyl radical; a guanidinium group ((R′)₂N—C(═NH₂⁺)—NR—) in which the radicals R and R′, independently of each other, represent a hydrogen atom or a C₁-C₄ alkyl radical;
   a nitro group; a cyano group; a halogen atom, preferably chlorine or fluorine;
   two radicals R₂ or two radicals R₃ or two radicals R₄, borne by adjacent carbon atoms, can form, with the carbon atom to which each is attached, a fused ring:
m represents an integer between 0 and 4;
m′ represents an integer between 0 and 4;
m″ represents an integer between 0 and 4;
e is an integer between 0 and 2;
the bond a derived from formula IIa, IIb, IIc, III or IV links the groups W₁, W₂ and W₃ to the azo group;
in the case of formulae IIa and IIc, and when two radicals R₂ borne by two adjacent carbon atoms form an aromatic ring, the bond a may link the group W₁ to the azo group via the said aromatic ring;
the bond a′ derived from formula IV links the groups W₃ to the linker L;
the electrical neutrality of the compounds being ensured by means of one or more cosmetically acceptable, identical or different anions An.

Everything that has been detailed previously regarding formula (I) obviously remains valid and will not be repeated in this part of the text.

The concentration of compounds of formula (I) may range between 0.001% and 5% by weight and preferably between about 0.05% and 2% by weight relative to the total weight of the dye composition.

The composition according to the invention may also comprise an oxidation base. This oxidation base may be chosen from the oxidation bases conventionally used in oxidation dyeing, for example para-phenylenediamines, bis(phenyl)alkylenediamines, para-aminophenols, ortho-aminophenols and heterocyclic bases.

Among the para-phenylenediamines that may be mentioned more particularly are, for example, para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxy-ethyl)-para-phenylenediamine, 4-N,N-bis(β-hydroxy-ethyl)amino-2-methylaniline, 4-N,N-bis(β-hydroxyethyl)-amino-2-chloroaniline, 2-β-hydroxyethyl-para-phenylene-diamine, 2-fluoro-para-phenylenediamine, 2-isopropyl-para-phenylenediamine, N-(β-hydroxypropyl)-para-phenylenediamine, 2-hydroxymethyl-para-phenylene-diamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylene-diamine, 2-β-acetylaminoethyloxy-para-phenylenediamine, N-(β-methoxyethyl)-para-phenylenediamine, 4-amino-phenylpyrrolidine, 2-thienyl-para-phenylenediamine, 2-β-hydroxyethylamino-5-aminotoluene, and the addition salts thereof with an acid.

Among the para-phenylenediamines mentioned above, para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the addition salts thereof with an acid, are particularly preferred.

Among the bis(phenyl)alkylenediamines that may be mentioned, for example, are N,N′-bis(β-hydroxy-ethyl)-N,N′-bis(4′-aminophenyl)-1,3-diaminopropanol, N,N′-bis(β-hydroxyethyl)-N,N′-bis(4′-aminophenyl)-ethylenediamine, N,N′-bis(4-aminophenyl)tetramethylene-diamine, N,N′-bis(β-hydroxyethyl)-N,N′-bis(4-amino-phenyl)tetramethylenediamine, N,N′-bis(4-methylamino-phenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the addition salts thereof with an acid.

Among the para-aminophenols that may be mentioned, for example, are para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethyl-aminomethyl)phenol and 4-amino-2-fluorophenol, and the addition salts thereof with an acid.

Among the ortho-aminophenols that may be mentioned, for example, are 2-aminophenol, 2-amino-5-methylphenol, 2-amino-6-methylphenol and 5-acetamido-2-aminophenol, and the addition salts thereof with an acid.

Among the heterocyclic bases that may be mentioned, for example, are pyridine derivatives, pyrimidine derivatives and pyrazole derivatives, and derivatives of pyrazolo[1,2-a]pyrazol-1-one type.

Among the pyridine derivatives that may be mentioned are the compounds described, for example, in patents GB 1 026 978 and GB 1 153 196, such as 2,5-diaminopyridine, 2-(4-methoxyphenyl)amino-3-amino-pyridine, 2,3-diamino-6-methoxypyridine, 2-(β-methoxy-ethyl)amino-3-amino-6-methoxypyridine and 3,4-diamino-pyridine, and the addition salts thereof with an acid.

Among the pyrimidine derivatives that may be mentioned are the compounds described, for example, in patents DE 2 359 399; JP 88-169 571; JP 05-163 124; EP 0 770 375 or patent application WO 96/15765, such as 2,4,5,6-tetraminopyrimidine, 4-hydroxy-2,5,6-triamino-pyrimidine, 2-hydroxy-4,5,6-triaminopyrimidine, 2,4-dihydroxy-5,6-diaminopyrimidine and 2,5,6-triamino-pyrimidine, and pyrazolopyrimidine derivatives such as those mentioned in patent application FR-A-2 750 048 and among which mention may be made of pyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo-[1,5-a]pyrimidin-5-ol; 2-(3-aminopyrazolo-[1,5-a]pyrimidin-7-ylamino)ethanol, 2-(7-amino-pyrazolo[1,5-a]pyrimidin-3-ylamino)ethanol, 2-[(3-aminopyrazolo[1,5-a]pyrimidin-7-yl)(2-hydroxyethyl)amino]ethanol, 2-[(7-aminopyrazolo[1,5-a]-pyrimidin-3-yl)(2-hydroxyethyl)amino]ethanol, 5,6-dimethylpyrazolo[1,5-a]pyrimidine-3,7-diamine, 2,6-dimethylpyrazolo[1,5-a]pyrimidine-3,7-diamine, 2,5,N7,N7-tetramethylpyrazolo[1,5-a]pyrimidine-3,7-diamine and 3-amino-5-methyl-7-imidazolylpropylamino-pyrazolo[1,5-a]pyrimidine, and the addition salts thereof with an acid and the tautomeric forms thereof, when a tautomeric equilibrium exists.

Among the pyrazole derivatives that may be mentioned are the compounds described in patents DE 3 843 892 and DE 4 133 957 and patent applications WO 94/08969, WO 94/08970, FR-A-2 733 749 and DE 195 43 988, such as 4,5-diamino-1-methylpyrazole, 4,5-diamino-1-(β-hydroxyethyl)pyrazole, 3,4-diamino-pyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenyl-pyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triamino-pyrazole, 3,5-diamino-1-methyl-4-methylaminopyrazole and 3,5-diamino-4-(β-hydroxyethyl)amino-1-methyl-pyrazole, and the addition salts thereof with an acid.

Among the derivatives of pyrazolo[1,2-a]pyrazol-1-one type, mention may be made of compounds such as 2,3-diamino-6,7-dihydro-1H,5H-pyrazolo[1,2-a]pyrazol-1-one.

The composition according to the invention may also contain one or more couplers conventionally used for dyeing keratin fibres. Among these couplers, mention may be made especially of meta-phenylene-diamines, meta-aminophenols, meta-diphenols, naphthalene-based couplers and heterocyclic couplers.

Examples that may be mentioned include 2-methyl-5-aminophenol, 5-N-(β-hydroxyethyl)amino-2-methylphenol, 6-chloro-2-methyl-5-aminophenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diamino-benzene, 1,3-bis(2,4-diaminophenoxy)propane, 3-ureido-aniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)-amino-3,4-methylenedioxybenzene and 2,6-bis(β-hydroxy-ethylamino)toluene and the addition salts thereof with an acid.

In the composition of the present invention, the coupler(s) is (are) generally present in an amount ranging from 0.001% to 10% by weight, and more preferably from 0.005% to 6% by weight, relative to the total weight of the dye composition. The oxidation base(s) is (are) present in an amount preferably ranging from 0.001% to 10% by weight, and more preferably from 0.005% to 6% by weight, relative to the total weight of the dye composition.

In general, the addition salts with an acid that may be used in the context of the dye compositions of the invention for the oxidation bases and couplers are chosen especially from the hydrochlorides, hydrobromides, sulfates, citrates, succinates, tartrates, lactates, tosylates, benzenesulfonates, phosphates and acetates.

The composition according to the invention may optionally comprise at least one additional direct dye other than the compounds of formula (I). This dye may be chosen from cationic and nonionic species.

Non-limiting examples that may be mentioned include nitrobenzene dyes, azo, azomethine, methine, tetraazapentamethine, anthraquinone, naphthoquinone, benzoquinone, phenothiazine, indigoid, xanthene, phenanthridine, phthalocyanin and triarylmethane-based dyes and natural dyes, alone or as mixtures.

It may be chosen, for example, from the following red or orange nitrobenzene dyes:
1-hydroxy-3-nitro-4-N-(γ-hydroxypropyl)amino-benzene,
N-(β-hydroxyethyl)amino-3-nitro-4-aminobenzene,
1-amino-3-methyl-4-N-(β-hydroxyethyl)amino-6-nitrobenzene,
1-hydroxy-3-nitro-4-N-(β-hydroxyethyl)amino-benzene,
1,4-diamino-2-nitrobenzene,
1-amino-2-nitro-4-methylaminobenzene,
N-(β-hydroxyethyl)-2-nitro-para-phenylenediamine,
1-amino-2-nitro-4-(β-hydroxyethyl)amino-5-chloro-benzene,
2-nitro-4-aminodiphenylamine,
1-amino-3-nitro-6-hydroxybenzene,
1-(β-aminoethyl)amino-2-nitro-4-(β-hydroxyethyl-oxy)benzene,
1-(β,γ-dihydroxypropyl)oxy-3-nitro-4-(β-hydroxy-ethyl)aminobenzene,
1-hydroxy-3-nitro-4-aminobenzene,
1-hydroxy-2-amino-4,6-dinitrobenzene,
1-methoxy-3-nitro-4-(β-hydroxyethyl)aminobenzene,
2-nitro-4′-hydroxydiphenylamine, and
1-amino-2-nitro-4-hydroxy-5-methylbenzene.

The additional direct dye may also be chosen from yellow and green-yellow nitrobenzene direct dyes; mention may be made, for example, of the compounds chosen from:
1-β-hydroxyethyloxy-3-methylamino-4-nitrobenzene,
1-methylamino-2-nitro-5-(β,γ-dihydroxypropyl)oxy-benzene,
1-(β-hydroxyethyl)amino-2-methoxy-4-nitrobenzene,
1-(β-aminoethyl)amino-2-nitro-5-methoxybenzene,
1,3-di(β-hydroxyethyl)amino-4-nitro-6-chloro-benzene,
1-amino-2-nitro-6-methylbenzene,
1-(β-hydroxyethyl)amino-2-hydroxy-4-nitrobenzene,
N-(β-hydroxyethyl)-2-nitro-4-trifluoromethyl-aniline,
4-(β-hydroxyethyl)amino-3-nitrobenzenesulfonic acid,
4-ethylamino-3-nitrobenzoic acid,
4-(β-hydroxyethyl)amino-3-nitrochlorobenzene,
4-(β-hydroxyethyl)amino-3-nitromethylbenzene,
4-(β,γ-dihydroxypropyl)amino-3-nitrotrifluoro-methylbenzene,
1-(β-ureidoethyl)amino-4-nitrobenzene,
1,3-diamino-4-nitrobenzene,
1-hydroxy-2-amino-5-nitrobenzene,
1-amino-2-[tris(hydroxymethyl)methyl]amino-5-nitrobenzene,
1-(β-hydroxyethyl)amino-2-nitrobenzene, and
4-(β-hydroxyethyl)amino-3-nitrobenzamide.

Mention may also be made of blue or violet nitrobenzene direct dyes, for instance:
1-(β-hydroxyethyl)amino-4-N,N-bis(β-hydroxyethyl)-amino-2-nitrobenzene,
1-(γ-hydroxypropyl)amino-4,N,N-bis(β-hydroxyethyl)-amino-2-nitrobenzene,
1-(β-hydroxyethyl)amino-4-(N-methyl-N-β-hydroxy-ethyl)amino-2-nitrobenzene,
1-(β-hydroxyethyl)amino-4-(N-ethyl-N-β-hydroxy-ethyl)amino-2-nitrobenzene,
1-(β,γ-dihydroxypropyl)amino-4-(N-ethyl-N-p-hydroxyethyl)amino-2-nitrobenzene,
2-nitro-para-phenylenediamines having the following formula:

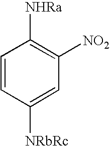

in which:
Rb represents a C₁-C₄ alkyl radical or a β-hydroxy-ethyl, β-hydroxypropyl or γ-hydroxypropyl radical;
Ra and Rc, which may be identical or different, represent a β-hydroxyethyl, β-hydroxypropyl, γ-hydroxypropyl or β,γ-dihydroxypropyl radical, at least one of the radicals Rb, Rc or Ra representing a γ-hydroxypropyl radical and Rb and Rc not simultaneously being able to denote a β-hydroxyethyl radical when Rb is a γ-hydroxypropyl radical, such as those described in French patent FR 2 692 572.

Among the azo direct dyes that may be used according to the invention, mention may be made of the cationic azo dyes described in patent applications WO 95/15144, WO 95/01772 and EP 714 954.

Among these compounds, mention may be made most particularly of the following dyes:
1,3-dimethyl-2-[[4-(dimethylamino)phenyl]azo]-1H-imidazolium chloride,
1,3-dimethyl-2-[(4-aminophenyl)azo]-1H-imidazolium chloride,
1-methyl-4-[(methylphenylhydrazono)methyl]-pyridinium methyl sulfate.

Among the azo direct dyes that may also be mentioned are the following dyes described in the Colour Index International 3rd edition:
Disperse Red 17
Acid Yellow 9
Acid Black 1
Basic Red 22
Basic Red 76
Basic Yellow 57
Basic Brown 16
Acid Yellow 36
Acid Orange 7
Acid Red 33
Acid Red 35
Basic Brown 17
Acid Yellow 23
Acid Orange 24
Disperse Black 9.

Mention may also be made of 1-(4′-aminodiphenylazo)-2-methyl-4-[bis(β-hydroxy-ethyl)amino]benzene and 4-hydroxy-3-(2-methoxyphenyl-azo)-1-naphthalenesulfonic acid.

Among the quinone direct dyes that may be mentioned are the following dyes:
Disperse Red 15
Solvent Violet 13
Acid Violet 43
Disperse Violet 1
Disperse Violet 4
Disperse Blue 1
Disperse Violet 8
Disperse Blue 3
Disperse Red 11
Acid Blue 62
Disperse Blue 7
Basic Blue 22
Disperse Violet 15
Basic Blue 99

and also the following compounds:
1-N-methylmorpholiniumpropylamino-4-hydroxy-anthraquinone
1-aminopropylamino-4-methylaminoanthraquinone
1-aminopropylaminoanthraquinone
5-β-hydroxyethyl-1,4-diaminoanthraquinone
2-aminoethylaminoanthraquinone
1,4-bis(β,γ-dihydroxypropylamino)anthraquinone.

Among the azine dyes that may be mentioned are the following compounds:
Basic Blue 17
Basic Red 2.

Among the triarylmethane dyes that may be used according to the invention, mention may be made of the following compounds:
Basic Green 1
Acid Blue 9
Basic Violet 3
Basic Violet 14
Basic Blue 7
Acid Violet 49
Basic Blue 26
Acid Blue 7.

Among the indoamine dyes that may be used according to the invention, mention may be made of the following compounds:
2-β-hydroxyethylamino-5-[bis(β-4′-hydroxyethyl)-amino]anilino-1,4-benzoquinone;
2-p-hydroxyethylamino-5-(2′-methoxy-4′-amino)-anilino-1,4-benzoquinone;
3-N(2′-chloro-4′-hydroxy)phenylacetylamino-6-methoxy-1,4-benzoquinoneimine;
3-N(3′-chloro-4′-methylamino)phenylureido-6-methyl-1,4-benzoquinoneimine;
3-[4′-N-(ethylcarbamylmethyl)amino]phenylureido-6-methyl-1,4-benzoquinoneimine.

Among the dyes of tetraazapentamethine type that may be used according to the invention, mention may be made of the following compounds given in the table below, An being defined as above:

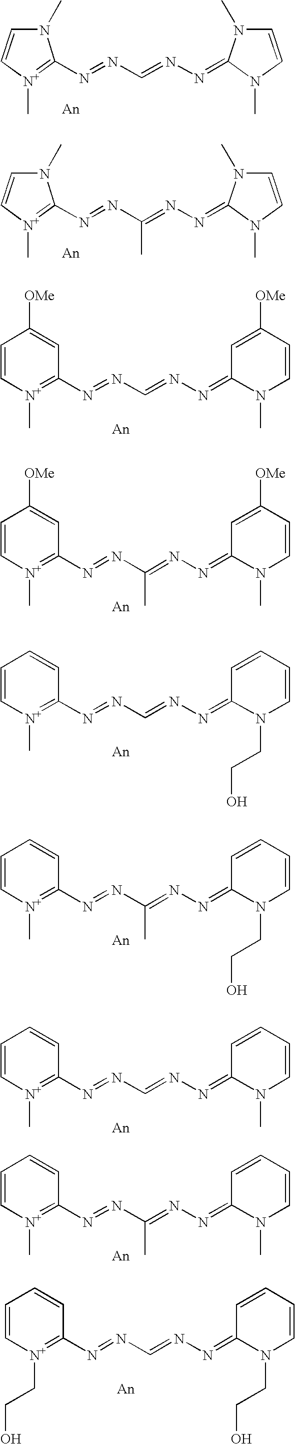

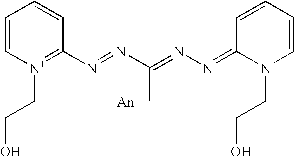

Among the natural direct dyes that may be used according to the invention, mention may be made of lawsone, juglone, alizarin, purpurin, carminic acid, kermesic acid, purpurogallin, protocatechaldehyde, indigo, isatin, curcumin, spinulosin and apigenidin. Extracts or decoctions containing these natural dyes may also be used, and especially henna-based poultices or extracts.

When they are present, the content of additional direct dye(s) in the composition generally ranges from 0.001% to 20% by weight relative to the weight of the composition and preferably from 0.01% to 10% by weight relative to the weight of the composition.

The medium that is suitable for dyeing, also known as the dye support, generally consists of water or of a mixture of water and of at least one organic solvent to dissolve the compounds that are not sufficiently water-soluble.

More particularly, the organic solvents are chosen from linear or branched, preferably saturated monoalcohols or diols containing from 2 to 10 carbon atoms, such as ethyl alcohol, isopropyl alcohol, hexylene glycol (2-methyl-2,4-pentanediol), neopentyl glycol and 3-methyl-1,5-pentanediol; aromatic alcohols such as benzyl alcohol and phenylethyl alcohol; glycols or glycol ethers, for instance ethylene glycol monomethyl, monoethyl and monobutyl ether, propylene glycol and its ethers, for instance propylene glycol monomethyl ether, butylene glycol and dipropylene glycol; and also diethylene glycol alkyl ethers, especially of C₁-C₄, for instance diethylene glycol monoethyl ether or monobutyl ether, alone or as a mixture.

The usual solvents described above, when they are present, usually represent from 1% to 40% by weight and more preferably from 5% to 30% by weight relative to the total weight of the composition.

The dye composition in accordance with the invention may also contain various adjuvants conventionally used in compositions for dyeing the hair, such as anionic, cationic, nonionic, amphoteric or zwitterionic surfactants or mixtures thereof, anionic, cationic, nonionic, amphoteric or zwitterionic polymers or mixtures thereof, mineral or organic thickeners, and in particular anionic, cationic, nonionic and amphoteric associative polymeric thickeners, antioxidants, penetrants, sequestrants, fragrances, buffers, dispersants, conditioning agents, for instance silicones, which may or may not be volatile or modified, film-forming agents, ceramides, preserving agents and opacifiers.

The above adjuvants are generally present in an amount for each of them of between 0.01% and 20% by weight relative to the weight of the composition.

Needless to say, a person skilled in the art will take care to select this or these optional additional compounds such that the advantageous properties intrinsically associated with the oxidation dye composition in accordance with the invention are not, or are not substantially, adversely affected by the envisaged addition(s).

The pH of the dye composition in accordance with the invention is generally between about 3 and 12 and preferably between about 5 and 11. It may be adjusted to the desired value using acidifying or basifying agents usually used in the dyeing of keratin fibres, or alternatively using standard buffer systems.

Among the acidifying agents that may be mentioned, for example, are mineral or organic acids such as hydrochloric acid, orthophosphoric acid, sulfuric acid, carboxylic acids such as acetic acid, tartaric acid, citric acid and lactic acid, and sulfonic acids.

Among the basifying agents that may be mentioned, for example, are aqueous ammonia, alkaline carbonates, alkanolamines such as monoethanolamine, diethanolamine and triethanolamine and derivatives thereof, sodium hydroxide, potassium hydroxide and the compounds having the following formula:

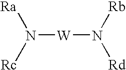

in which W is a propylene residue optionally substituted with a hydroxyl group or a C₁-C₄ alkyl radical; Rₐ, R_{b}, R_{c} and R_{d}, which may be identical or different, represent a hydrogen atom, a C₁-C₄ alkyl radical or a C₁-C₄ hydroxyalkyl radical.

The dye composition according to the invention may be in various forms, such as in the form of liquids, creams or gels, or in any other form that is suitable for dyeing keratin fibres, and especially human hair.

The composition according to the invention may also comprise at least one oxidizing agent. In this case, the composition is referred to as a ready-to-use composition.

For the purposes of the present invention, the term “ready-to-use composition” means a composition intended to be applied immediately to the keratin fibres, i.e. it may be stored in unmodified form before use or may result from the extemporaneous mixing of two or more compositions.

Usually, the said composition is obtained by mixing the composition according to the invention with an oxidizing composition.

The oxidizing agent may be any oxidizing agent conventionally used in the field. Thus, it may be chosen from hydrogen peroxide, urea peroxide, alkali metal bromates, persalts such as perborates and persulfates, and also enzymes, among which mention may be made of peroxidases, 2-electron oxidoreductases such as uricases, and 4-electron oxygenases, for instance laccases. The use of hydrogen peroxide is particularly preferred.

The content of oxidizing agent is generally between 1% and 40% by weight relative to the weight of the ready-to-use composition, and preferably between 1% and 20% by weight relative to the weight of the ready-to-use composition.

Generally, the oxidizing composition used is an aqueous composition and may be in the form of a solution or an emulsion.

Usually, the composition free of oxidizing agent is mixed with about 0.5 to 10 weight equivalents of the oxidizing composition.

It should be noted that the pH of the ready-to-use composition is more particularly between 4 and 12 and preferably between 7 and 11.5.

The pH of the composition may be adjusted using an acidifying or basifying agent chosen especially from those mentioned previously in the context of the description according to the invention.

A subject of the invention is also a dyeing process that includes the application of a dye composition according to the invention to wet or dry keratin fibres.

The application to the fibres of the dye composition comprising the compound(s) of formula (I) or the addition salts thereof with an acid, optionally at least one oxidation base optionally combined with at least one coupler, and optionally at least one additional direct dye, may be performed in the presence of an oxidizing agent.

This oxidizing agent may be added to the composition comprising the compound(s) of formula (I) and the optional oxidation bases, couplers and/or additional direct dyes, either at the time of use or directly onto the keratin fibre.

The oxidizing composition may also contain various adjuvants conventionally used in compositions for dyeing the hair and as defined above.

The pH of the oxidizing composition containing the oxidizing agent is such that, after mixing with the dye composition, the pH of the resulting composition applied to the keratin fibres preferably ranges between 4 and 12 approximately and even more preferably between 7 and 11.5. It may be adjusted to the desired value by means of acidifying or basifying agents usually used in the dyeing of keratin fibres and as defined above.

The composition that is finally applied to the keratin fibres may be in various forms, such as in the form of liquids, creams or gels or in any other form that is suitable for dyeing keratin fibres, more particularly human keratin fibres and especially the hair.

According to one particular embodiment, the composition according to the invention is free of oxidation base and of coupler.

The composition applied may optionally comprise at least one oxidizing agent.

The composition is thus applied to the wet or dry keratin fibres and is then left for a leave-in time that is sufficient to obtain the desired coloration.

Whatever the variant adopted (with or without oxidizing agent) the leave-in time is generally between a few seconds and 30 minutes and preferably between 3 and 15 minutes.

The temperature at which the composition is left to act is generally between 15 and 220° C., more particularly between 15 and 80° C. and preferably between 15 and 40° C.

After the leave-in time, the composition is removed by rinsing with water, optionally followed by washing with a shampoo, and then optionally drying.

Another subject of the invention is a multi-compartment device or dyeing “kit” in which a first compartment contains the dye composition of the invention and a second compartment contains the oxidizing composition. This device may be equipped with a means for applying the desired mixture to the hair, such as the devices described in patent FR-2 586 913.

The examples that follow serve to illustrate the invention without, however, being limiting in nature.

### EXAMPLE 1

Dye 1 is synthesized according to the third embodiment. The implementation conditions of a synthesis of this kind were summarized earlier on.

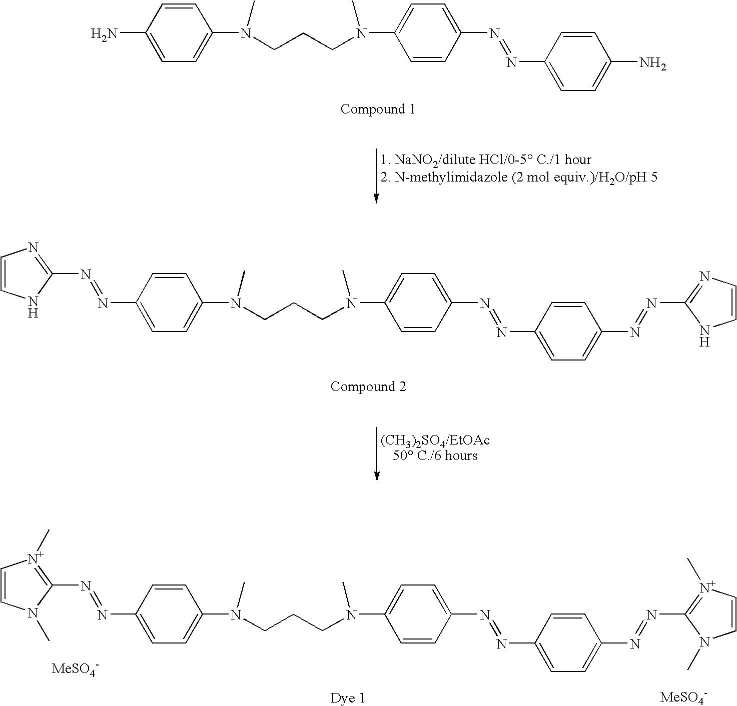

Compound 1 is brought into contact with NaNO₂ and dilute hydrochloric acid at a temperature of between 0 and 5° C. for one hour.

Then 2 mol equivalents of N-methylimidazole are added and the pH is adjusted to 5 by adding aqueous sodium hydroxide solution, and compound 2 is recovered.

The product is separated by filtration.

This compound 2 is subsequently contacted with (CH₃)₂SO₄ in ethyl acetate at 50° C. for 6 hours. At the outcome of the reaction, dye 1 is recovered.

Dye 1 is recovered by filtration.

### Example of Dyeing:

| Dye 1 | 2.5 × 10⁻⁴ mol |
|---|---|
| Dye support (1) | (*) |
| Demineralized water qs (g) | 100 |

(*) Dye support

| | | |
|---|---|---|
| 96° ethyl alcohol | 20.8 | g |
| C₈-C₁₀ alkyl polyglucoside as an aqueous 60% solution | 12 | g |
| Benzyl alcohol | 4.0 | g |
| Polyethylene glycol containing 8 ethylene oxide units | 6.0 | g |
| Na₂HPO₄ | 0.28 | g |
| KH₂PO₄ | 0.46 | g |

The mixture obtained is applied to locks of grey hair containing 90% white hairs. After leaving in for 20 minutes, the locks of hair are rinsed, washed with a standard shampoo, rinsed again and then dried.

A chestnut shade is obtained and the colour is shampoo-fast and light-fast.

1. Dissymmetrical cationic polyazo compounds of formula (I) below:
Dye1-L-Dye2
in which formula:
Dye1 represents: 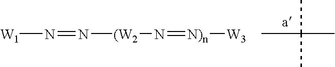
Dye2 represents: 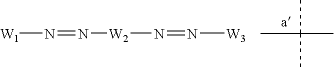
in which:
W₁, which may be identical or different, represent a heteroaromatic radical chosen from the following formulae: 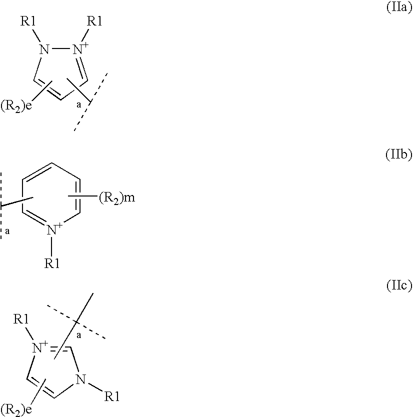
W₂, which may be identical or different, represent a carbon-based aromatic group having the following formula: 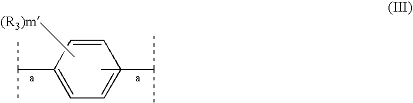
W₃, which may be identical or different, represent a carbon-based aromatic group having the following formula: 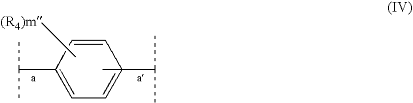
L represents a C₁-C₆₀, preferably C₂-C₄₀ and even more particularly C₂-C₂₀ saturated or unsaturated, linear or branched, cyclic or non-cyclic, (hetero)aromatic or non-(hetero)aromatic, optionally substituted hydrocarbon-based chain, optionally bearing at least one cationic charge, optionally interrupted with at least one hetero atom or group comprising at least one hetero atom, preferably oxygen, nitrogen or sulfur, L not comprising any diazo, nitro, nitroso or peroxide groups; L may be terminated at one and/or the other of its ends with at least one hetero atom or group comprising at least one hetero atom, preferably oxygen, nitrogen or sulfur;
n is 0 or 1;
R₁, independently of each other, represent:
a linear or branched, saturated or unsaturated, aromatic or non-aromatic, optionally substituted C₁-C₁₆ hydrocarbon-based chain, which can form one or more 3- to 7-membered carbon-based rings, optionally interrupted with one or more hetero atoms chosen from oxygen, nitrogen and sulfur; R₁ not comprising any nitro, nitroso, peroxide or diazo bonds R₁ is directly attached to the nitrogen atom (quaternized or non-quaternized) of the heteroaromatic ring via a carbon atom;
R₂, R₃ and R₄, independently of each other, represent:
a linear or branched, saturated or unsaturated, aromatic or non-aromatic, optionally substituted C₁-C₁₆ hydrocarbon-based chain, which can form one or more 3- to 6-membered carbon-based rings, optionally interrupted with one or more hetero atoms or with one or more groups bearing at least one hetero atom, preferably chosen from oxygen and nitrogen;
a hydroxyl group,
a C₁-C₄ alkoxy group,
a C₂-C₄ (poly)hydroxyalkoxy group; an alkoxycarbonyl group (RO—CO—) in which R represents a C₁-C₄ alkyl radical, an alkylcarbonyloxy radical (RCO—O—) in which R represents a C₁-C₄ alkyl radical;
an amino group, an amino group substituted with one or more C₁-C₄ alkyl radicals, which may be identical or different, optionally bearing at least one hydroxyl group, the two alkyl radicals possibly forming, with the nitrogen atom to which they are attached, a 5- or 6-membered heterocycle optionally bearing another hetero atom identical to or different from nitrogen, for example oxygen or sulfur; an alkylcarbonylamino group (RCO—NR—) in which the radicals R, independently of each other, represent a C₁-C₄ alkyl radical; a carbamoyl group ((R)₂N—CO) in which the radicals R, independently of each other, which may be identical or different, represent a hydrogen atom or a C₁-C₄ alkyl radical; a ureido group ((R)₂N—CO—NR′—) in which the radicals R and R′, independently of each other, represent a hydrogen atom or a C₁-C₄ alkyl radical; a sulfonamide group ((R)₂N—SO₂—) in which the radicals R, independently of each other, represent a hydrogen atom or a C₁-C₄ alkyl radical; an alkylsulfonylamino group (RSO₂—NR′—) in which the radicals R and R′, independently of each other, represent a hydrogen atom or a C₁-C₄ alkyl radical; a guanidinium group ((R′)₂N—C(═NH₂⁺)—NR—) in which the radicals R and R′, independently of each other, represent a hydrogen atom or a C₁-C₄ alkyl radical;
a nitro group; a cyano group; a halogen atom, preferably chlorine or fluorine;
two radicals R₂ or two radicals R₃ or two radicals R₄, borne by adjacent carbon atoms, can form, with the carbon atom to which each is attached, a fused ring:
m represents an integer between 0 and 4;
m′ represents an integer between 0 and 4;
m″ represents an integer between 0 and 4;
e is an integer between 0 and 2;
the bond a derived from formula IIa, IIb, IIc, III or IV links the groups W₁, W₂ and W₃ to the azo group;
in the case of formulae IIa and IIc, and when two radicals R₂ borne by two adjacent carbon atoms form an aromatic ring, the bond a may link the group W₁ to the azo group via the said aromatic ring;
the bond a′ derived from formula IV links the group W₃ to the linker L;
the electrical neutrality of the compounds being ensured by means of one or more cosmetically acceptable, identical or different anions An.

2. Compounds according to the preceding claim, characterized in that the radicals R₁, independently of each other, represent a C₁-C₁₅ alkyl radical; a C₂-C₆ monohydroxyalkyl radical; a C₂-C₆ polyhydroxyalkyl radical; a (C₁-C₆)alkoxy(C₂-C₆)alkyl radical; an optionally substituted aryl radical, such as phenyl; an optionally substituted arylalkyl radical, such as benzyl; a C₂-C₆ amidoalkyl radical; a C₂-C₆ aminoalkyl radical in which the amine is substituted with two optionally substituted C₁-C₄ alkyl radicals, which may be identical or different; the radical R₁ being linked to the nitrogen atom via a carbon atom.

3. Compounds according to either of claims 1 and 2, characterized in that the radicals R₁, independently of each other, represent a C₁-C₆ alkyl radical, a C₂-C₆ monohydroxyalkyl radical; a C₂-C₆ polyhydroxyalkyl radical; a (C₁-C₆)alkoxy(C₂-C₆)alkyl radical; a phenyl radical optionally substituted with at least one chlorine atom, a hydroxyl group, a group RCO—NH— in which R represents a C₁-C₄ alkyl radical, an amino radical substituted with two C₁-C₄ alkyl radicals, which may be identical or different; a benzyl radical; a C₁-C₆ aminoalkyl radical; a C₁-C₆ aminoalkyl radical in which the amine is substituted with two C₁-C₄ alkyl radicals, which may be identical or different; the radical R₁ being linked to the nitrogen atom via a carbon atom.

4. Compounds according to any one of the preceding claims, characterized in that the radicals R₂, R₃ and R₄, independently of each other, more particularly represent:
a hydrogen atom;
an optionally substituted C₁-C₁₆ and preferably C₁-C₈ alkyl radical;
a halogen atom such as chlorine, fluorine or bromine;
a hydroxyl group;
a C₁-C₂ alkoxy radical; a C₂-C₄ (poly)hydroxyalkoxy radical;
an amino radical, an amino radical substituted with one or two C₁-C₄ alkyl radicals, which may be identical or different, optionally bearing at least one hydroxyl or C₁-C₄ dialkylamino group, the two alkyl radicals possibly forming, with the nitrogen atom to which they are attached, a saturated or unsaturated, optionally substituted 5- or 6-membered heterocycle optionally bearing another hetero atom identical to or different from nitrogen;
an alkylcarbonylamino radical (RCO—NR′—) in which the radical R represents a C₁-C₄ alkyl radical and the radical R′ represents a hydrogen atom or a C₁-C₄ alkyl radical; a carbamoyl radical ((R)₂N—CO—) in which the radicals R, which may be identical or different, represent a hydrogen atom or a C₁-C₄ alkyl radical optionally bearing at least one hydroxyl group; an alkylsulfonylamino radical (R′SO₂—NR—) in which the radical R represents a hydrogen atom or a C₁-C₄ alkyl radical optionally bearing at least one hydroxyl group, and the radical R′ represents a C₁-C₄ alkyl radical; an aminosulfonyl radical ((R)₂N—SO₂—) in which the radicals R, which may be identical or different, represent a hydrogen atom or a C₁-C₄ alkyl radical optionally bearing at least one hydroxyl group; a ureido radical (N(R)₂—CO—NR′—) in which the radicals R and R′, which may be identical or different, represent a hydrogen atom or a C₁-C₄ alkyl radical.

5. Compounds according to any one of the preceding claims, characterized in that the radicals R₂, R₃ and R₄, independently of each other, represent:
a hydrogen atom;
a C₁-C₄ alkyl radical optionally substituted with one or more radicals chosen from hydroxyl, acylamino and amino radicals substituted with two identical or different C₁-C₂ alkyl radicals, optionally bearing at least one hydroxyl group, or a C₁-C₂ alkoxy radical;
an amino radical; an amino radical substituted with one or two C₁-C₃ alkyl radicals, which may be identical or different, optionally bearing at least one hydroxyl group, the two alkyl radicals possibly forming, with the nitrogen atom to which they are attached, a saturated or unsaturated, optionally substituted 5- or 6-membered heterocycle optionally bearing another hetero atom identical to or different from nitrogen; an acylamino radical; a carbamoyl radical; a sulfonylamino radical;
a hydroxyl radical; a C₁-C₂ alkoxy radical;
a chlorine atom.

6. Compounds according to any one of the preceding claims, characterized in that L represents a chain that isolates each of the chromophores so as to stop the electron delocalization of each of the chromophores.

7. Compounds according to any one of the preceding claims, characterized in that L represents the following formula: 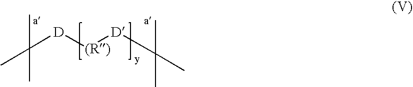
in which:
D and D′, independently of each other, represent a linear or branched C₁-C₁₄ hydrocarbon-based bond, which can form one or more 5- to 8-membered carbon-based rings, and which may be saturated or unsaturated, optionally interrupted with one or more hetero atoms or with one or more groups bearing at least one hetero atom, preferably chosen from oxygen and nitrogen; D and/or D′ are linked to the ammonium atom via a carbon atom;
the bond a′ links the arms D and D′ to the groups W₃, preferably via an oxygen atom or a nitrogen atom;
y is greater than or equal to 1 and preferably equal to 1 or 2;
the group R″ representing 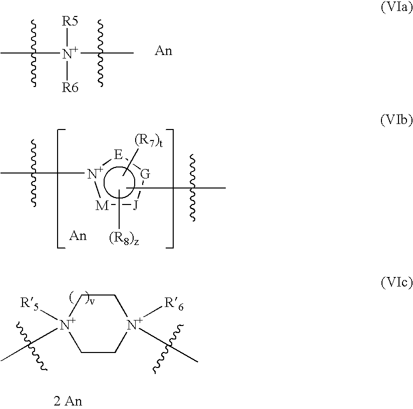
R₅, R₆, R′₅ and R′₆, independently of each other, represent a C₁-C₁₅ alkyl radical; a C₁-C₆ monohydroxyalkyl radical; a C₂-C₆ polyhydroxyalkyl radical; a (C₁-C₆)alkoxy(C₁-C₆)alkyl radical; an aryl radical such as phenyl; an arylalkyl radical such as benzyl; a C₁-C₆ amidoalkyl radical; a C₁-C₆ aminoalkyl radical; a C₁-C₆ aminoalkyl radical in which the amine is substituted with one or more identical or different C₁-C₄ alkyl, (C₁-C₆)alkylcarbonyl, acylamino or (C₁-C₆)alkylsulfonyl radicals;
R₅ and R₆ may form, together with the nitrogen atom to which they are attached, a 5-, 6- or 7-membered saturated cationic ring that may contain one or more hetero atoms, the cationic ring possibly being substituted with a halogen atom, a hydroxyl radical, a C₁-C₆ alkyl radical, a C₁-C₆ monohydroxyalkyl radical, a C₂-C₆ polyhydroxyalkyl radical, a C₁-C₆ alkoxy radical, an amido radical, a (C₁-C₆)alkyl-carbonyl radical, a thio radical, a C₁-C₆ thioalkyl radical, a (C₁-C₆)alkylthio radical, an amino radical or an amino radical substituted with one or more identical or different C₁-C₆ alkyl, (C₁-C₆)alkyl-carbonyl, acylamino or (C₁-C₆)alkylsulfonyl radicals; and preferably unsubstituted;
R₅ or R₆ may form, with D or D′, a 5-, 6- or 7-membered saturated cationic ring that may contain one or more hetero atoms, the cationic ring possibly being substituted with a halogen atom, a hydroxyl radical, a C₁-C₆ alkyl radical, a C₁-C₆ monohydroxy-alkyl radical, a C₂-C₆ polyhydroxyalkyl radical, a C₁-C₆ alkoxy radical, an amido radical, a (C₁-C₆)alkyl-carbonyl radical, a thio radical, a C₁-C₆ thioalkyl radical, a (C₁-C₆)alkylthio radical, an amino radical or an amino radical substituted with one or more identical or different C₁-C₆ alkyl, (C₁-C₆)alkyl-carbonyl, acylamino or (C₁-C₆)alkylsulfonyl radicals; and preferably unsubstituted;
the ring members E, G, J and M, which may be identical or different, represent a carbon, oxygen, sulfur or nitrogen atom to form a pyrazolium, imidazolium, triazolium, oxazolium, isoxazolium, thiazolium or isothiazolium ring,
R₇ represents a C₁-C₆ alkyl radical; a C₁-C₆ mono-hydroxyalkyl radical, a C₂-C₆ polyhydroxyalkyl radical, a (C₁-C₆)alkoxy(C₁-C₆)alkyl radical, a C₂-C₆ carbamylalkyl radical, a (C₁-C₆)alkylcarboxy(C₁-C₆)alkyl radical or a benzyl radical; it being understood that the radical R₇ is borne by a nitrogen atom,
R₈, which may be identical or different, represent a hydrogen or halogen atom, a C₁-C₆ alkyl radical, a C₁-C₆ monohydroxyalkyl radical, a C₂-C₆ polyhydroxyalkyl radical, a hydroxyl radical, a C₁-C₆ alkoxy radical, an amido radical, a carboxyl radical, a C₁-C₆ alkylcarbonyl radical, a C₁-C₆ thioalkyl radical, a (C₁-C₆)alkylthio radical, an amino radical disubstituted with a (C₁-C₆)alkyl, (C₁-C₆)alkylcarbonyl or (C₁-C₆)alkylsulfonyl radical, a benzyl radical, a phenyl radical optionally substituted with one or more radicals chosen from methyl, hydroxyl, amino and methoxy radicals, it being understood that the radicals R₈ are borne by a carbon atom,
An represents one or more organic or mineral anions;
z is an integer between 1 and 3;
t is equal to 0 or 1; when t=0, then D′ is linked to the nitrogen atom via a carbon atom,
v is an integer equal to 1 or 2,
y is equal to 0 or 1.

8. Compounds according to the preceding claim, characterized in that, in formulae (VIa) and (VIc), R₅, R₆, R′₅ and R′₆, independently of each others represent a C₁-C₆ alkyl radical, a C₁-C₄ monohydroxyalkyl radical, a C₂-C₄ polyhydroxyalkyl radical, a (C₁-C₆)alkoxy(C₂-C₄)alkyl radical, a C₂-C₆ amidoalkyl radical or a C₂-C₆ dimethylaminoalkyl radical.

9. Compounds according to the preceding claim, characterized in that R₅, R₆, R′₅ and R′₆, independently of each other, represent a methyl, ethyl or 2-hydroxyethyl radical.

10. Compounds according to any one of claims 7 to 9, characterized in that D and D′, separately, are preferably a C₁-C₆ alkyl chain that may be substituted, and that is preferably unsubstituted.

11. Compounds according to claim 7, characterized in that, in formula (VIb), the ring members E, G, J and M form an imidazolium, pyrazolium, oxazolium or thiazolium ring.

12. Compounds according to the preceding claim, characterized in that y and t are equal to 0.

13. Compounds according to either of claims 11 and 12, characterized in that D and D′ represent a C₁-C₆ alkyl chain that may be substituted, and that is preferably unsubstituted.

14. Compounds according to any one of claims 1 to 7, characterized in that L is represented by a (C₁-C₂₀)alkyl-(hetero)aryl-(C₁-C₂₀)alkyl group.

15. Compounds according to the preceding claim, characterized in that L is represented by a (C₁-C₁₀)alkyl-(hetero)aryl-(C₁-C₁₀)alkyl group.

16. Compounds according to either of claims 14 and 15, characterized in that the (hetero)arylene radicals are chosen from phenylene or naphthylene, phenanthrylene, triazinyl, pyrimidinyl, pyridyl, pyridazinyl and quinoxalinyl.

17. Compounds according to any one of claims 14 to 16, characterized in that among the alkylene radicals interrupted with a (hetero)arylene group, mention may be made of the following radicals: 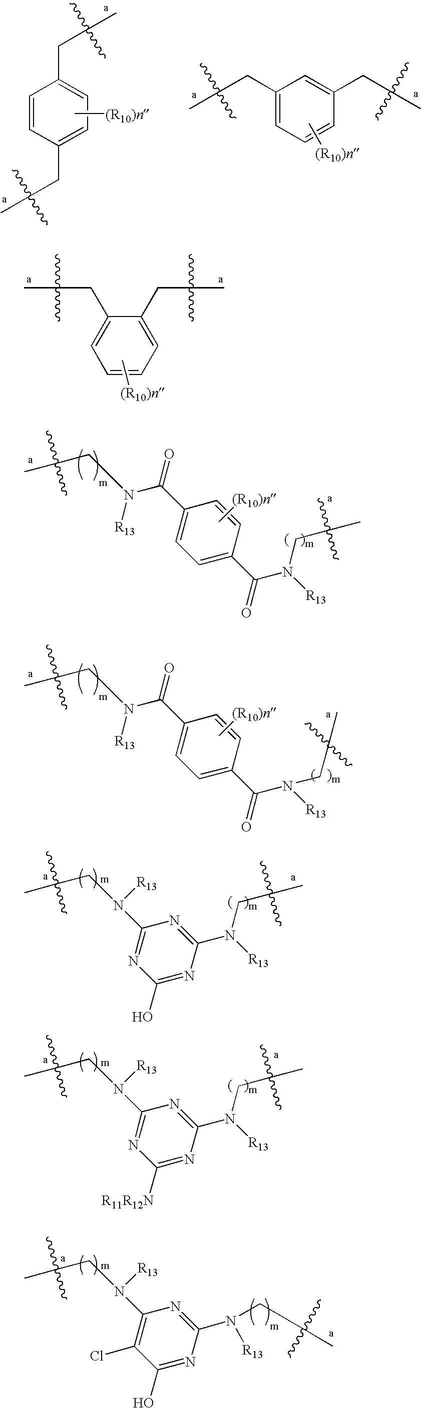 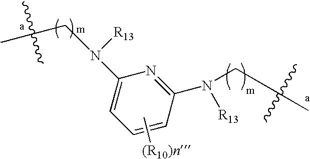
in which:
R₁₀ has the same definition as R₄,
R₁₁ and R₁₂ represent, independently of each other, a hydrogen atom or a C₁-C₈ alkyl radical, optionally substituted with one or more hydroxyl, C₁-C₂ alkoxy, C₂-C₄ (poly)hydroxyalkoxy, amino, C₁-C₂ (di)alkylamino or optionally substituted aryl radicals,
R₁₃ represent, independently of each other, a C₁-C₄ alkyl radical,
m represents an integer between 1 and 6,
n″ represents an integer between 0 and 4,
n′″ represents an integer between 0 and 3.

18. Compounds according to any one of the preceding claims, characterized in that the cosmetically acceptable anion is chosen from halides such as chlorides, bromides, fluorides or iodides; hydroxides; sulfates; hydrogen sulfates; (C₁-C₆)alkyl sulfates; phosphates; carbonates; hydrogen carbonates, perchlorates; acetates; tartrates; citrates, oxalates; (C₁-C₆)alkylsulfonates such as methylsulfonate; arylsulfonates that are unsubstituted or substituted with a C₁-C₄ alkyl radical, for instance a 4-tolylsulfonate.

19. Compounds according to any one of the preceding claims, characterized in that they correspond to the following formulae: 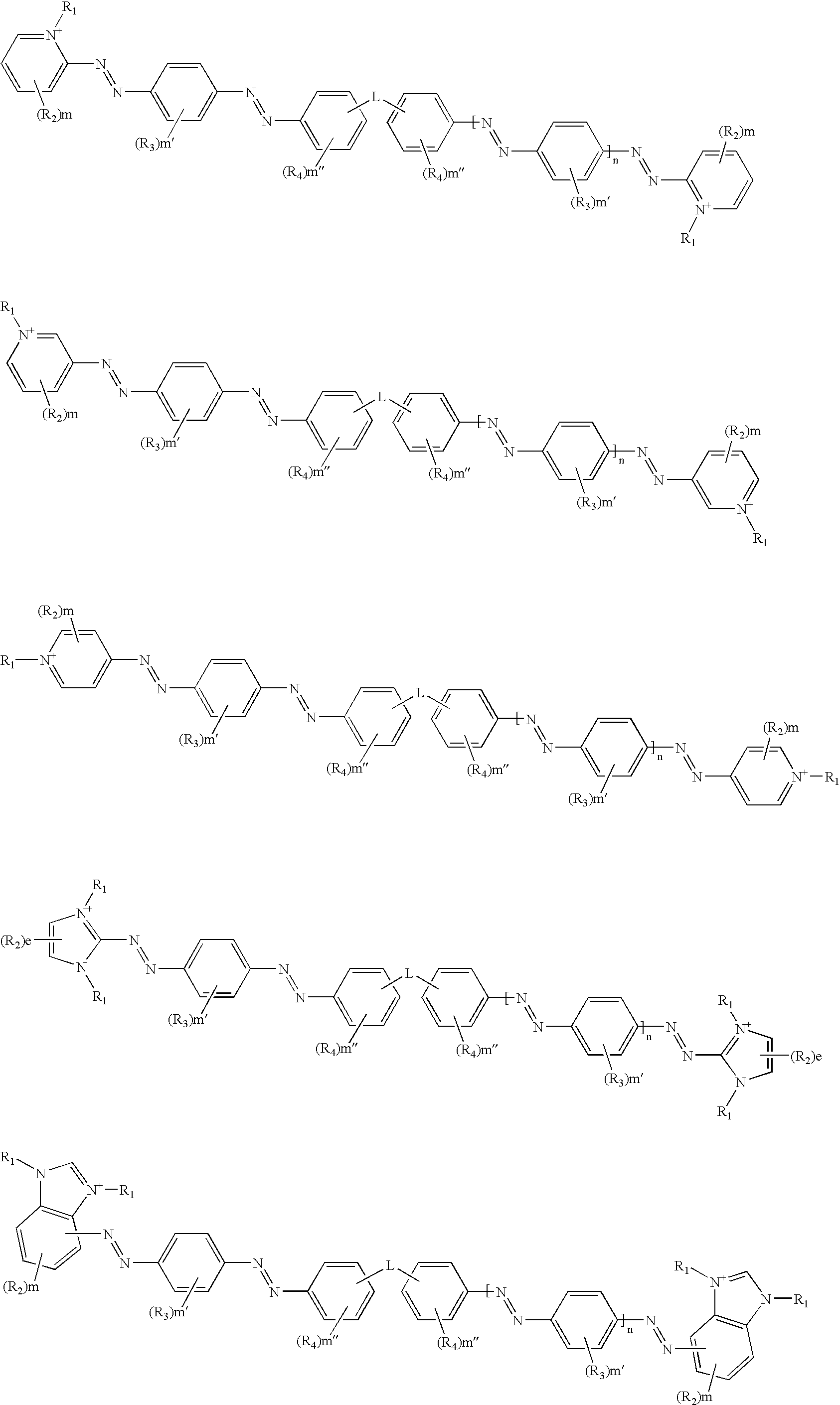 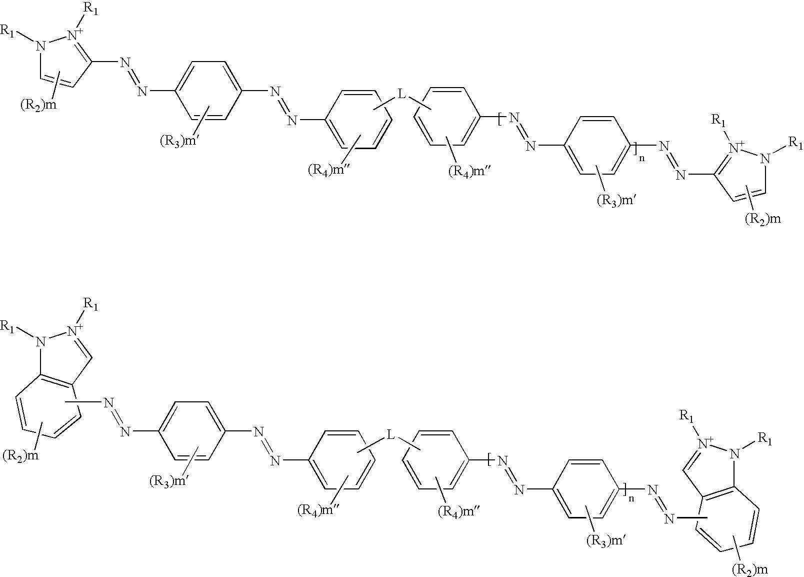
in which formulae:
R₁, R₂, R₃, R₄, n and m have been defined above;
the electrical neutrality of the molecule being respected by means of the presence of one or more cosmetically acceptable anions An as defined above.

20. Dye composition comprising, in a medium that is suitable for dyeing keratin fibres, as direct dye, at least one dissymmetrical cationic polyazo compound of formula (I) below:
Dye1-L-Dye1
in which formula:
Dye1 represents: 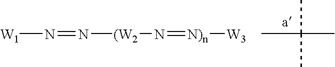
Dye2 represents: 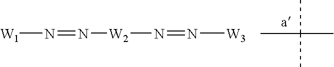
in which:
W₁, which may be identical or different, represent a heteroaromatic radical chosen from the following formulae: 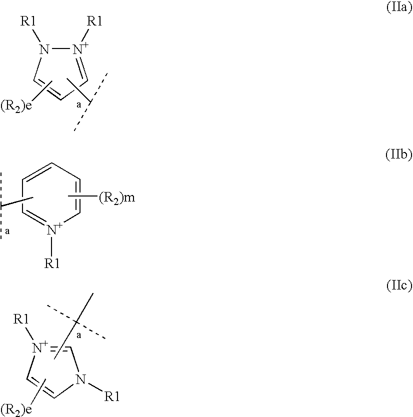
W₂, which may be identical or different, represent a carbon-based aromatic group having the following formula: 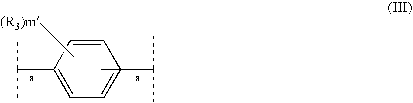
W₃, which may be identical or different, represent a carbon-based aromatic group having the following formula: 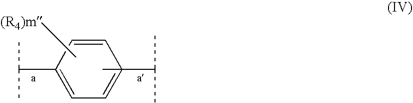
L represents a C₁-C₆₀, preferably C₂-C₄₀ and even more particularly C₂-C₂₀ saturated or unsaturated, linear or branched, cyclic or non-cyclic, (hetero)aromatic or non-(hetero)aromatic, optionally substituted hydrocarbon-based chain, optionally bearing at least one cationic charge, optionally interrupted with at least one hetero atom or group comprising at least one hetero atom, preferably oxygen, nitrogen or sulfur, L not comprising any diazo, nitro, nitroso or peroxide groups; L may be terminated at one and/or the other of its ends with at least one hetero atom or group comprising at least one hetero atom, preferably oxygen, nitrogen or sulfur;
n is equal to 0 or 1;
R₁, independently of each other, represent:
a linear or branched, saturated or unsaturated, aromatic or non-aromatic, optionally substituted C₁-C₁₆ hydrocarbon-based chain, which can form one or more 3- to 7-membered carbon-based rings, optionally interrupted with one or more hetero atoms chosen from oxygen, nitrogen and sulfur; R₁ not comprising any nitro, nitroso, peroxide or diazo bonds. R₁ is directly attached to the nitrogen atom (quaternized or non-quaternized) of the heteroaromatic ring via a carbon atom;
R₂, R₃ and R₄, independently of each other, represent:
a linear or branched, saturated or unsaturated, aromatic or non-aromatic, optionally substituted C₁-C₁₆ hydrocarbon-based chain, which can form one or more 3- to 6-membered carbon-based rings, optionally interrupted with one or more hetero atoms or with one or more groups bearing at least one hetero atom, preferably chosen from oxygen and nitrogen;
a hydroxyl group,
a C₁-C₄ alkoxy group,
a C₂-C₄ (poly)hydroxyalkoxy group; an alkoxycarbonyl group (RO—CO—) in which R represents a C₁-C₄ alkyl radical, an alkylcarbonyloxy radical (RCO—O—) in which R represents a C₁-C₄ alkyl radical;
an amino group, an amino group substituted with one or more C₁-C₄ alkyl radicals, which may be identical or different, optionally bearing at least one hydroxyl group, the two alkyl radicals possibly forming, with the nitrogen atom to which they are attached, a 5- or 6-membered heterocycle optionally bearing another hetero atom identical to or different from nitrogen, for example oxygen or sulfur; an alkylcarbonylamino group (RCO—NR—) in which the radicals R, independently of each other, represent a C₁-C₄ alkyl radical; a carbamoyl group ((R)₂N—CO) in which the radicals R, independently of each other, which may be identical or different, represent a hydrogen atom or a C₁-C₄ alkyl radical; a ureido group ((R)₂N—CO—NR′—) in which the radicals R and R′, independently of each other, represent a hydrogen atom or a C₁-C₄ alkyl radical; a sulfonamide group ((R)₂N—SO₂—) in which the radicals R, independently of each other, represent a hydrogen atom or a C₁-C₄ alkyl radical; an alkylsulfonylamino group (RSO₂—NR′—) in which the radicals R and R′, independently of each other, represent a hydrogen atom or a C₁-C₄ alkyl radical; a guanidinium group ((R′)₂N—C(═NH₂⁺)—NR—) in which the radicals R and R′, independently of each other, represent a hydrogen atom or a C₁-C₄ alkyl radical;
a nitro group; a cyano group; a halogen atom, preferably chlorine or fluorine;
two radicals R₂ or two radicals R₃ or two radicals R₄, borne by adjacent carbon atoms, can form, with the carbon atom to which each is attached, a fused ring:
m represents an integer between 0 and 4;
m′ represents an integer between 0 and 4;
m″ represents an integer between 0 and 4;
e is an integer between 0 and 2;
the bond a derived from formula IIa, IIb, IIc, III or IV links the groups W₁, W₂ and W₃ to the azo group;
in the case of formulae IIa and IIc, and when two radicals R₂ borne by two adjacent carbon atoms form an aromatic ring, the bond a may link the group W₁ to the azo group via the said aromatic ring;
the bond a′ derived from formula IV links the group W₃ to the linker L;
the electrical neutrality of the compounds being ensured by means of one or more cosmetically acceptable, identical or different anions An.

21. Composition according to the preceding claim, characterized in that the content of compound of formula (I) represents from 0.001% to 5% by weight and preferably from 0.05% to 2% by weight relative to the weight of the composition.

22. Composition according to either of claims 20 and 21, characterized in that it comprises at least one additional direct dye, at least one oxidation base or at least one coupler.

23. Composition according to the preceding claim, characterized in that the additional direct dye is a cationic or nonionic dye chosen from nitrobenzene dyes, azo, azomethine, methine, tetraazapentamethine, anthraquinone, naphthoquinone, benzoquinone, phenothiazine, indigoid, xanthene, phenanthridine, phthalocyanin and triarylmethane-based dyes, and natural dyes, alone or as mixtures.

24. Composition according to claim 22, characterized in that the oxidation base is chosen from p-phenylenediamines, bis(phenyl)alkylenediamines, o-aminophenols, p-aminophenols and heterocyclic bases, and the addition salts thereof with an acid.

25. Composition according to claim 22, characterized in that the coupler is chosen from m-aminophenols, m-phenylenediamines, m-diphenols, naphthols and heterocyclic couplers, and the addition salts thereof with an acid.

26. Composition according to any one of claims 21 to 25, characterized in that it comprises at least one oxidizing agent.

27. Process for dyeing keratin fibres, which consists in placing a composition according to any one of claims 21 to 26 in contact with the said wet or dry fibres, for a time that is sufficient to obtain the desired effect.

28. Multi-compartment device in which a first compartment contains the composition according to any one of claims 21 to 26 and a second compartment contains an oxidizing composition.
